(19) United States
(12) Patent Application Publication
(10) Pub. No.: US 2026/0069695 A1
(43) Pub. Date: Mar. 12, 2026

(54) COMPOSITIONS AND METHODS FOR TREATING AUTOIMMUNE DISEASES WITH ANTI-CLL-1 DIRECTED THERAPY

(71) Applicant: Sovereign, Middleman, and Riley LLC, New York, NY (US)

(72) Inventor: Matt Mandel, New York, NY (US)

(21) Appl. No.: 19/324,059

(22) Filed: Sep. 9, 2025

Related U.S. Application Data
(60) Provisional application No. 63/692,182, filed on Sep. 9, 2024.

(51) Int. Cl. A61K 40/22; A61K 40/11; A61K 40/31; A61K 40/41; C07K 14/725; C07K 16/24

(52) U.S. Cl. CPC A61K 40/22; A61K 40/11; A61K 40/31; A61K 40/416; C07K 14/7051; C07K 16/24; C07K 2317/622

(57) ABSTRACT

Disclosed are methods of treating autoimmune diseases by administering engineered T cells expressing a chimeric antigen receptor (CAR) that specifically binds C-type lectin-like molecule-1 (CLL-1). The CAR-T cells deplete CLL-1-expressing monocyte-derived macrophages that replace yolk sac-derived or other embryonically derived tissue-resident macrophages, thereby reducing inflammation and disease progression. The methods are applicable to multiple sclerosis and other autoimmune disorders.

### CROSS-REFERENCE TO RELATED APPLICATIONS

This application claims the benefit of priority to U.S. Provisional Patent Application No. 63/692,182 filed on Sep. 9, 2024, the entire contents of which are incorporated herein by reference in their entirety.

### FIELD OF INVENTION

The present disclosure generally relates to the field of immunotherapy, and more particularly to methods and compositions for treating CLL-1-expressing myeloid cell-mediated autoimmune diseases and disorders, specifically multiple sclerosis, using treatment modalities that target C-type lectin-like molecule-1 (CLL-1) expressing border-associated macrophages.

### BACKGROUND

Multiple sclerosis (MS) is a chronic autoimmune disease characterized by inflammation, demyelination, and neurodegeneration in the central nervous system (CNS). The disease affects approximately 2.8 million people worldwide and is a leading cause of disability in young adults. MS typically manifests with a relapsing-remitting course that may eventually transition to a progressive phase.

Current therapeutic approaches for MS primarily focus on modulating adaptive immune responses, particularly targeting T and B lymphocytes. These treatments include disease-modifying therapies such as interferon-beta, glatiramer acetate, sphingosine-1-phosphate receptor modulators, anti-CD20 antibodies, and various immunosuppressants. While these therapies have shown efficacy in reducing relapse rates they have limited effectiveness in progressive forms of the disease.

The pathophysiology of MS involves complex interactions between the peripheral immune system and the CNS. The blood-brain barrier (BBB) normally restricts the entry of immune cells into the CNS. However, in MS, this barrier becomes compromised and contributes to inflammation and tissue damage.

Recent research has highlighted the role of innate immune cells, particularly myeloid cells such as macrophages, in MS pathogenesis. Border-associated macrophages (BAMs) are a specialized population of tissue-resident macrophages located at CNS interfaces, including the choroid plexus, meninges, and perivascular spaces. These cells express various pattern recognition receptors and can respond to both pathogen-associated molecular patterns and damage-associated molecular patterns.

BAMs have been implicated in neuroinflammatory processes and may contribute to what has been termed “smoldering inflammation” in MS. This chronic, low-grade inflammation persists despite conventional immunomodulatory therapies and is thought to drive progressive neurodegeneration. The concept of smoldering inflammation represents a paradigm shift in understanding MS pathogenesis, suggesting that compartmentalized immune responses within the CNS, rather than peripheral immune cell infiltration, may be responsible for ongoing tissue damage in progressive forms of the disease.

Molecular markers expressed by BAMs have emerged as potential therapeutic targets. Various C-type lectin receptors are expressed on myeloid cells and can mediate both pro-inflammatory and anti-inflammatory responses depending on the context. These receptors recognize carbohydrate structures and can influence cellular activation, phagocytosis, and cytokine production.

Biomarkers for monitoring MS disease activity and treatment response remain an area of active investigation. Conventional markers include magnetic resonance imaging (MRI) findings, relapse rates, and disability progression measures such as the Expanded Disability Status Scale (EDSS). However, these markers have limitations in sensitivity and specificity, particularly for detecting subtle changes in disease activity or predicting long-term outcomes.

Cerebrospinal fluid (CSF) biomarkers offer potential advantages for monitoring CNS-specific processes. Established CSF biomarkers in MS include oligoclonal bands, immunoglobulin G index, and neurofilament light chain. Additional biomarkers reflecting myeloid cell activation or neuroinflammation could provide complementary information about disease mechanisms and treatment effects.

The development of cellular therapies for autoimmune diseases represents an evolving field. Chimeric antigen receptor (CAR) T cell therapy, which has shown remarkable success in certain hematological malignancies, is being explored for autoimmune conditions. This approach involves genetically engineering T cells to express receptors targeting specific antigens, potentially allowing for selective depletion of pathogenic cell populations while sparing beneficial immune functions.

Challenges in applying CAR-T cell therapy to autoimmune diseases include identifying appropriate target antigens, managing potential off-target effects, ensuring adequate trafficking to sites of inflammation, and maintaining therapeutic efficacy over time. Various strategies to enhance CAR-T cell function and safety are under investigation, including modifications to reduce immunogenicity, prevent exhaustion, and improve persistence in vivo.

The unmet need in MS treatment, particularly for progressive forms, underscores the importance of developing novel therapeutic approaches targeting disease mechanisms not adequately addressed by current therapies. Strategies focusing on innate immune components, especially tissue-resident myeloid cells, may offer new avenues for intervention in this complex neurological disorder.

Multiple sclerosis (MS) is a debilitating autoimmune disorder characterized by chronic inflammation and demyelination within the central nervous system (CNS). The disease manifests in various forms, including relapsing-remitting MS (RRMS), secondary progressive MS (SPMS), and primary progressive MS (PPMS), each differing in symptoms and disease progression. Current therapeutic strategies primarily focus on reducing acute inflammatory episodes and managing symptoms rather than addressing the underlying chronic inflammation that contributes to progressive neurological decline.

One of the critical challenges in treating MS is the smoldering neuroinflammation that persists even during periods of clinical remission. This low-level inflammation is difficult to treat effectively with existing therapies.

The role of border-associated macrophages (BAMs) in MS has recently become a focus of research due to their strategic location at key interfaces within the CNS, such as the choroid plexus, meninges, and perivascular spaces. By residing at these borders, BAMs regulate immune cell entry and interstitial fluid exchange, positioning them as critical mediators of chronic CNS inflammation. However, the targeting of BAMs has not been adequately explored in existing therapeutic modalities for MS, representing a gap in the current treatment landscape.

In summary, the treatment of MS faces several challenges, including inadequate targeting of smoldering neuroinflammation. These issues underscore the need for novel therapeutic strategies that can address these unmet needs by effectively targeting the specific immune components involved in the chronic aspects of the disease.

Multiple sclerosis (MS) remains one of the most challenging neurological disorders to manage effectively, particularly due to its various forms and phases. The disease primarily involves an immune-mediated process in which an abnormal response of the body's immune system is directed against the central nervous system (CNS), comprising the brain, spinal cord, and optic nerves. Within the CNS, the immune system causes inflammation that damages myelin—the substance that surrounds and insulates the nerves—as well as the nerves themselves, and the specialized cells that make myelin.

Relapsing-remitting MS (RRMS) is the most common disease course and is characterized by clearly defined attacks of worsening neurologic function. These attacks are followed by partial or complete recovery periods. Secondary progressive MS (SPMS) and primary progressive MS (PPMS) involve a steady progression of disability. In all types, smoldering neuroinflammation is a critical factor that contributes to the continuous progression of the disease, often independently of relapses.

Current therapeutic interventions for MS primarily involve immunomodulatory and immunosuppressive drugs that aim to reduce the frequency and severity of relapses, and to a lesser extent, slow the progression of disability. These therapies, however, do not adequately or fully halt this progression nor specifically address the underlying smoldering inflammation that leads to ongoing neurological deterioration.

The etiology for many autoimmune disorders and diseases is unknown. CLEC12A (also known as C-type lectin-like molecule-1, CLL-1), a member of the C-type lectin receptor family, is expressed on myeloid cells, including monocytes and macrophages, border associated macrophages (BAMs), and BAM-like macrophages. BAMs are a specialized subset of macrophages located at the interfaces of the CNS, including the meninges, perivascular spaces, and choroid plexus. These cells play critical roles in maintaining CNS homeostasis when in the healthy state. The same cells, however, become pathogenic in autoimmune and MS settings, where they shift from maintaining homeostasis to driving Neuro inflammation, supporting survival of auto reactive, lymphocytes, and contributing directly to immune mediated demyelination. Emerging evidence suggests that BAMs, including choroid plexus macrophages (CPMs), express CLL-1 at higher levels when diseased than they do in the healthy state, making them potential targets for novel immunotherapeutic approaches.

Accordingly, there remains a need for effective treatments that can target the inflammation mediated pathological immune cells in autoimmune disease and disorders such as MS, thereby addressing both relapsing and progressive forms of the disease. Furthermore, there is need for improved monitoring methods of neuroinflammation and MS disease activity, particularly during relapses and in progressive stages of the disease. These needs and other needs are met by the various aspects of the present disclosure.

### SUMMARY

Conventional models of multiple sclerosis have described inflammation and neurodegeneration as largely independent processes, without connecting immune checkpoint biology to the clinical transition from relapsing to progressive disease. It is described herein that CCR2+ CLL-1+ monocyte-derived macrophages are a source of inhibitory ligands that suppress CD-8+ T-cell activity and thereby drive the transition from an acute-inflammatory, relapsing MS course to a chronic inflammatory progressive MS course. This mechanistic link between cellular pathology and the natural history of MS has not previously been described in the art. Accordingly, selective depletion of these monocyte-derived macrophages with CLL-1-directed CAR-T cells represents a novel therapeutic strategy distinct from oncologic CAR-T approaches (e.g., AML) or B-cell-directed therapies in MS (e.g., anti-CD19, anti-BCMA).

Under conditions of inflammation, pathogenic CCR2+ CLL-1+ monocyte-derived macrophages (BAM-like macrophages) are recruited into the central nervous system (CNS), via border regions including the choroid plexus, meninges, and perivascular spaces where they replace the homeostatic yolk-sac derived border associated macrophages present since birth. These recruited BAM-like macrophages localize within compartments that are contiguous with interstitial fluid pathways, positioning them in direct proximity to perivascular lymphocytes that accumulate in the same fluid-trafficking niches. Treatment with CLL-1-directed CAR-T cells depletes these CLL-1-expressing BAM-like macrophages, thereby reducing chronic compartmentalized neuroinflammation in multiple sclerosis.

Analogous to chronic lymphocytic leukemia (CLL), in which nurse-like cells and other monocyte-derived macrophages provide pro-survival signals to malignant B cells, pathogenic CCR2+ CLL-1+ monocyte-derived macrophages in multiple sclerosis adopt a similar role at central nervous system (CNS) borders. These cells replace homeostatic yolk sac-derived border-associated macrophages and sustain autoreactive B cells within perivascular and meningeal immune niches, thereby supporting the persistence of tertiary lymphoid structures and compartmentalized neuroinflammation. Accordingly, depletion of CLL-1-expressing monocyte-derived macrophages by CAR-T cells disrupts the B cell survival axis, interrupting a pathogenic mechanism in MS that directly parallels the tumor-supportive microenvironment of B-cell leukemias.

Although conventional models of multiple sclerosis pathology emphasize perivascular lymphocyte cuffs and suggest that the blood-brain barrier is the principal entry site for both B & T lymphocytes in the disease, evidence simultaneously indicates that the blood-CSF barrier at the choroid plexus constitutes a major immunological gateway for T cells. Activated CD4* and CD8+ T lymphocytes can traffic into the CNS through the choroid plexus, facilitated by upregulation of adhesion molecules such as VCAM-1 and ICAM-1 on choroid plexus epithelium and by chemokine gradients including CCL20. Both experimental autoimmune encephalomyelitis models and human pathological studies demonstrate T-cell migration across the choroid plexus epithelium into the CSF. At this site, fenestrated capillaries permit plasma filtrate into the stroma, which is processed by the epithelial layer to form CSF. This interstitial fluid is contiguous with the fluid in perivascular spaces and drains into meningeal lymphatic vessels, where it becomes lymph, thereby explaining the frequent observation of perivascular lymphocyte cuffing. Adoptively transferred CAR-T cells follow these same trafficking routes, gaining direct access to CNS border compartments where CCR2* CLL-1* monocyte-derived macrophages reside. This insight provides a mechanistic rationale for the ability of CLL-1 directed CAR-T cells to reach and deplete CCR2+ CLL-1+ monocyte derived macrophages in these fluid contiguous compartments.

Calprotectin (S100A8/A9) is a damage-associated molecular pattern (DAMP) released by activated monocytes and macrophages, functioning as both a biomarker and a driver of pathogenic inflammation. Its upregulation amplifies its own expression and induces CCL2, thereby sustaining the recruitment and activation of CCR2* CLL-1* monocyte-derived macrophages. As described above, in multiple sclerosis, these recruited macrophages replace yolk sac-derived border-associated macrophages and establish pathogenic niches at the choroid plexus, meninges, and perivascular spaces. Accordingly, cerebrospinal fluid calprotectin serves as a mechanistic biomarker of monocyte/macrophage activation within these fluid-contiguous compartments, directly reflecting the activity of the CLL-1* myeloid populations targeted by anti-CLL-1+ CAR-T therapy. In certain embodiments, calprotectin levels may also be used to monitor therapeutic efficacy of any intervention that targets or affects CCR2* CLL-1* monocyte-derived macrophages, including but not limited to CAR-T cells, monoclonal antibodies, small molecules such as TYK2 inhibitors, or other immunomodulatory agents, with measurement performed in CSF, blood, or other biological samples by ELISA, mass spectrometry, or equivalent assays.

In various aspects, provided herein are methods and compositions for treating CLL-1 (CLEC12A)-expressing myeloid cell-mediated autoimmune diseases and disorders. In further aspects, the CLL-1-expressing myeloid cell-mediated autoimmune diseases and disorders may be MS or lupus. In still further aspects, the CLL-1-expressing myeloid cell may be border associated macrophages (BAMs) and BAM-like macrophages, such as choroid plexus macrophages (CPMs).

In various aspects, provided herein are methods and compositions for treating CLL-1 expressing myeloid cell-mediated autoimmune diseases and disorders. In further aspects, the CLL-1-expressing myeloid cell-mediated autoimmune diseases and disorders may be MS. In still further aspects, the CLL-1-expressing myeloid cell may be a monocyte-derived BAM-like macrophage, such as choroid plexus macrophages (CPMs).

In one aspect, provided herein are methods and compositions for treating multiple sclerosis by targeting C-type lectin-like molecule-1 (CLL-1) expressed on border associated macrophages (BAMs) and other immune cells involved in neuroinflammation. For example, treatment with CLL-1-directed CAR-T cells depletes CLL-1-expressing monocyte derived macrophages thereby reducing compartmentalized neuroinflammation in multiple sclerosis.

In another aspect, provided herein are methods of treating a CLL-1-expressing myeloid cell-mediated autoimmune disease or disorder in a subject, comprising administering to the subject a therapeutically effective amount of engineered CAR-T cells that express a chimeric antigen receptor (CAR) targeting CLL-1, wherein the CAR-T cells target CLL-1 expressing BAM-like macrophages.

In another aspect, provided herein are methods of depleting CLL-1-expressing BAM-like macrophages in a subject with multiple sclerosis, comprising administering to the subject a therapeutically effective amount of engineered CAR-T cells that express a CAR targeting CLL-1.

In another aspect, provided herein are methods of reducing smoldering neuroinflammation in a subject with multiple sclerosis, comprising administering to the subject a therapeutically effective amount of engineered CAR-T cells that express a CAR targeting CLL-1.

In another aspect, provided herein are engineered CAR-T cells comprising a CAR targeting CLL-1 and at least one genetic modification selected from the group consisting of T cell receptor (TCR) knockout, programmed cell death protein 1 (PD-1) knockout, beta-2-microglobulin (B2M) knockout, and B2M-HLA-E insertion.

In another aspect, provided herein are methods of monitoring response to CLL-1-targeted therapy in a subject with multiple sclerosis, comprising measuring calprotectin levels in cerebrospinal fluid before and after administration of CAR-T cells targeting CLL-1.

In another aspect, provided herein are pharmaceutical compositions and kits comprising engineered CAR-T cells that express a CAR targeting CLL-1 for use in treating multiple sclerosis.

In another aspect, also provided herein are systems and kits comprising the disclosed methods and compositions. These and other aspects, objects, features, and advantages will become more apparent when read with the detailed description and figures provided herein.

### BRIEF DESCRIPTION OF THE DRAWINGS

The accompanying drawings illustrate several embodiments of the disclosed method and compositions and together with the description, serve to explain the principles of the disclosed method and compositions.

FIG. 1 depicts an anti-CLL-1 CAR-T cell therapeutic engaging a macrophage involved in the multiple sclerosis disease process via binding to CLL-1 expressed on the macrophage's surface.

FIG. 2 depicts the fenestrated capillary endothelium of the choroid plexus, showing the route through which the CAR-T therapeutic will travel to engage its target.

FIG. 3 illustrates the locations of border-associated macrophages (BAMs) in the spinal cord and surrounding areas, showing their expression of various markers including CLL-1.

FIG. 4 shows a close-up view of BAMs located perivascularly, highlighting their position within the blood-brain barrier in both acute and chronic lesions.

FIG. 5 depicts perivascular lymphocyte cuffing, a hallmark pathological finding in multiple sclerosis, with BAMs located within the area of perivascular cuffing.

FIG. 6 provides a microscopic image showing the histology of perivascular lymphocyte cuffing in multiple sclerosis.

FIG. 7 illustrates the comparison between early and advanced multiple sclerosis, highlighting the role of perivascular macrophages in disease progression.

FIG. 8 shows a close-up view of border-associated macrophages located in the choroid plexus, illustrating their expression of CLL-1 and other markers.

FIG. 9 illustrates a spinal tap procedure used for cerebrospinal fluid sampling for calprotectin measurement.

### DETAILED DESCRIPTION

The following detailed description is presented to enable any person skilled in the art to make and use the disclosed subject matter. Various modifications to the illustrated embodiments will be readily apparent to those skilled in the art, and the generic principles defined herein may be applied to other embodiments and applications without departing from the spirit and scope of the disclosed subject matter. Thus, the presently disclosed subject matter is not intended to be limited to the embodiments shown, but is to be accorded the widest scope consistent with the principles and features disclosed herein. The present disclosure is intended to cover alternatives, modifications, and equivalents, which may be included within the scope of the present disclosure.

The embodiments and examples described herein are illustrative only and not intended to be limiting in any way. Various alternatives may be envisioned by those with skill in the art. These alternatives may be used in implementing the present technology in accordance with a particular application. The headings provided herein are for convenience only and do not necessarily affect the scope or meaning of the claimed invention.

Unless defined otherwise, technical and scientific terms used herein have the same meaning as commonly understood by one of ordinary skill in the art to which this invention belongs. Sec, e.g. Singleton et al., Dictionary of Microbiology and Molecular Biology 2nd ed., J. Wiley & Sons (New York, N. Y. 1994); Sambrook et al., Molecular Cloning, A Laboratory Manual, Cold Spring Harbor Press (Cold Spring Harbor, NY 1989). All publications, patents and patent applications mentioned in this specification are incorporated by reference in their entirety for all purposes to the same extent as if each individual publication, patent, or patent application was specifically and individually indicated to be incorporated by reference.

### Definitions

As used herein, the terms “a,” “an,” and “the” include plural referents unless the context clearly dictates otherwise. The term “or” refers to a single element of stated alternative elements or a combination of two or more elements, unless the context clearly indicates otherwise. As used in the specification and the appended claims, the singular forms “a,” “an,” and “the” include plural referents unless the context clearly dictates otherwise. The term “and/or” includes any and all combinations of one or more of the associated listed items.

The terms “comprising,” “including,” “having,” and the like are synonymous and are used inclusively, in an open-ended fashion, and do not exclude additional elements, features, acts, operations, and so forth. The term “or” is used in its inclusive sense (and not in its exclusive sense) so that when used, for example, to connect a list of elements, the term “or” means one, some, or all of the elements in the list.

As used herein, the term “multiple sclerosis” or “MS” refers to a chronic autoimmune disease characterized by demyelination and neurodegeneration in the central nervous system. MS encompasses various clinical subtypes, including relapsing-remitting MS (RRMS), secondary progressive MS (SPMS), and primary progressive MS (PPMS).

As used herein, the term “acute neuroinflammation” refers to inflammatory processes in MS characterized by peripheral autoreactive T and B cells crossing the blood-brain barrier, leading to inflammatory lesions and relapses.

As used herein, the term “smoldering neuroinflammation” refers to inflammatory processes in MS driven by CNS-resident immune cells, including border-associated macrophages, which contribute to progressive neurodegeneration independent of peripheral immune cell infiltration.

As used herein, the term “border-associated macrophage” or “BAM” refers to CNS-resident monocyte-derived cells located at the interfaces between the CNS and periphery, including the choroid plexus, meninges, and perivascular spaces. BAMs express various markers, including CD206, CD63, CD45, FOLR2, P2RX7, NRP1, MHC II, and CLL-1 (CLEC12A).

As used herein, the term “choroid plexus macrophage” or “CPM” refers to a subset of BAMs located in the choroid plexus that express CLL-1 (CLEC12A) and play a role in neuroinflammation.

As used herein, the term “C-type lectin-like molecule-1” or “CLL-1” or “CLEC12A” refers to a type II transmembrane glycoprotein expressed myeloid cells, including dendritic cells and monocytes and macrophages, border associated macrophages (BAMs), and BAM-like macrophages on BAMs, including CPMs.

As used herein, the term “CLEC12A-expressing myeloid cell-mediated autoimmune diseases and disorders” refers to diseases or conditions in which the pathological or clinical manifestations are caused, driven, or substantially perpetuated by myeloid lineage cells (e.g., monocytes, macrophages, granulocytes, or dendritic cells) that express CLEC12A (also known as CLL-1). These autoimmune diseases or disorders are characterized by a dysregulated or maladaptive immune response directed against self-antigens, where CLEC12A-expressing myeloid cells play a significant role in initiating, sustaining, or exacerbating the inflammatory and tissue-damaging processes underlying the disease. This term is intended to encompass any autoimmune pathology in which such CLEC12A-expressing cells substantially contribute to disease onset, progression, or clinical symptoms.

As used herein, the term “chimeric antigen receptor” or “CAR” refers to an engineered receptor that combines an antigen-recognition domain with T cell signaling domains, enabling T cells to recognize and eliminate cells expressing a specific target antigen.

As used herein, the term “CAR-T cell” refers to a T cell engineered to express a chimeric antigen receptor.

As used herein, the term “CB-012” refers to engineered CAR-T cells that express an anti-CLL-1 chimeric antigen receptor and may include additional genetic modifications such as TCR knockout, PD-1 knockout, B2M knockout, and B2M-HLA-E insertion.

As used herein, the term “T cell receptor knockout” or “TCR knockout” refers to the genetic elimination of the endogenous T cell receptor in engineered CAR-T cells to prevent graft-versus-host disease and improve CAR-T cell specificity.

As used herein, the term “programmed cell death protein 1 knockout” or “PD-1 knockout” refers to the genetic elimination of the PD-1 immune checkpoint receptor in engineered CAR-T cells to prevent T cell exhaustion.

As used herein, the term “beta-2-microglobulin knockout” or “B2M knockout” refers to the genetic elimination of beta-2-microglobulin in engineered CAR-T cells to prevent expression of classical MHC class I molecules, reducing recognition by host cytotoxic T cells.

As used herein, the term “B2M-HLA-E insertion” refers to the genetic addition of beta-2-microglobulin fused to HLA-E in engineered CAR-T cells to prevent natural killer (NK) cell-mediated killing while maintaining the benefit of B2M knockout.

As used herein, the term “calprotectin” refers to a calcium-binding protein complex composed of S100A8 and S100A9 that is released by activated macrophages and may serve as a biomarker for monitoring treatment efficacy in MS.

As used herein, the term “gene” refers to a nucleic acid (e.g., DNA or RNA) sequence that including coding sequences necessary for the production of a polypeptide, RNA (e.g., including, but not limited to, mRNA, tRNA and rRNA) or precursor. The polypeptide, RNA, or precursor can be encoded by a full-length coding sequence or by any portion thereof. The term also encompasses the coding region of a structural gene and the sequences located adjacent to the coding region on both the 5′ and 3′ ends for a distance of about 1 kb on either end such that the gene corresponds to the length of the full-length mRNA. The term “gene” encompasses both cDNA and genomic forms of a gene, which may be made of DNA, or RNA. A genomic form or clone of a gene may contain the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation.

A “protein coding sequence” or a sequence that encodes a particular protein or polypeptide, is a nucleic acid sequence that is transcribed into mRNA (in the case of DNA) and is translated (in the case of mRNA) into a polypeptide in vitro or in vivo when placed under the control of appropriate regulatory sequences. The boundaries of the coding sequence are determined by a start codon at the 5′ terminus (N-terminus) and a translation stop nonsense codon at the 3′ terminus (C-terminus). A coding sequence can include, but is not limited to, cDNA from prokaryotic or eukaryotic mRNA, genomic DNA sequences from prokaryotic or eukaryotic DNA, and synthetic nucleic acids. A transcription termination sequence will usually be located 3′ to the coding sequence.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides may be isolated from genes, or chemically synthesized by methods known in the art. The terms “polynucleotide” and “nucleic acid” should be understood to include, as applicable to the embodiments being described, single-stranded (such as sense or antisense) and double-stranded polynucleotides.

The term “construct” refers to a recombinant genetic molecule having one or more isolated polynucleotide sequences. Genetic constructs used for transgene expression in a host organism include a series of cassettes including units with (in the 5′-3′ direction), a promoter sequence; a sequence encoding a gene of interest; and a termination sequence. The construct may also include selectable marker gene(s) and other regulatory elements for expression.

The term “expression” as used herein refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

As used herein, the term “inhibit” or other forms of the word such as “inhibiting” or “inhibition” means to hinder or restrain a particular characteristic, for example, to reduce, decrease or prevent, either partially or entirely. It is understood that this is typically in relation to some standard or expected value, in other words it is relative, but that it is not always necessary for the standard or relative value to be referred to. For example, “inhibits BAMs” means hindering or restraining the activity of the cell relative to a standard or a control. “Inhibits BAMs” can also mean to reduce or deplete the population of the cell relative to a standard or control.

As used herein, the terms “optional” or “optionally” means that the subsequently described event or circumstance can or cannot occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. Unless noted otherwise, the exemplary subject referred to in this disclosure is human. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects. In some aspects of the disclosed methods, the subject has been diagnosed with a need for treatment of one or more disorders prior to the administering step. In some aspects of the disclosed methods, the subject has been diagnosed with a need for treatment of one or more autoimmune disorders or diseases prior to the administering step. In some aspects of the disclosed method, the subject has been diagnosed with an inflammatory disorder or disease prior to the administering step. In some aspects of the disclosed method, the subject been diagnosed with multiple sclerosis prior to the administering step.

As used herein, the term “treatment” or “treating” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed. For example, preventing autoimmune disease or disorder means reducing the incidences, delaying or reversing diseases or disorders that are related to or associated with autoimmune pathology in which CLEC12A-expressing cells substantially contribute to disease onset, progression, or clinical symptoms.

As used herein, the term “diagnosed” means having been subjected to a physical examination by a person of skill, for example, a physician, and found to have a condition that can be diagnosed or treated by the compounds, compositions, or methods disclosed herein. In some aspects of the disclosed methods, the subject has been diagnosed with a need for treatment of an autoimmune disorder or disease prior to the administering step.

As used herein, the phrase “identified to be in need of treatment for a disorder,” or the like, refers to selection of a subject based upon need for treatment of the disorder. It is contemplated that the identification can, in one aspect, be performed by a person different from the person making the diagnosis. It is also contemplated, in a further aspect, that the administration can be performed by one who subsequently performed the administration.

As used herein, the term “providing” refers to any method of administering or contacting a disclosed compound, composition, or treatment to a cell, target receptor, or other biological entity, preparation to a subject. Such methods are well known to those skilled in the art and include, but are not limited to, oral administration, transdermal administration, administration by inhalation, nasal administration, topical administration, intravaginal administration, ophthalmic administration, intrathecal administration, intra-aural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The term “contacting” as used herein refers to bringing a disclosed composition, compound, or treatment and a cell, target receptor, or other biological entity together in such a manner that the composition or compound can affect the activity of the target (e.g., receptor, cell, etc.), either directly; i.e., by interacting with the target itself, or indirectly; i.e., by interacting with another molecule, co-factor, factor, or protein on which the activity of the target is dependent.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

Recitation of ranges of values herein are merely intended to serve as a shorthand method of referring individually to each separate value falling within the range, unless otherwise indicated herein, and each separate value is incorporated into the specification as if it were individually recited herein.

The use of any and all examples, or exemplary language (e.g., “such as”) provided herein, is intended merely to better illuminate the description and does not pose a limitation on the scope of the description unless otherwise claimed.

All methods described herein can be performed in any suitable order unless otherwise indicated or otherwise clearly contradicted by context. The use of any and all examples, or exemplary language (e.g., “such as”) provided herein, is intended merely to better illuminate the embodiments and does not pose a limitation on the scope of the embodiments unless otherwise claimed. No language in the specification should be construed as indicating any non-claimed element as essential to the practice of the invention.

According to various aspects of the present disclosure, and without being bound by a particular theory, the inventor has reduced to practice, as disclosed in the various embodiments, key biological insights relevant to autoimmune diseases and disorders, and particularly, multiple sclerosis pathogenesis.

In various embodiments, the invention can involve pathogenic lymphocyte trafficking. In further aspects, B cells and T cells that accumulate in perivascular spaces are not primarily crossing the blood-brain barrier. Instead, they can arrive through interstitial fluid trafficking from fenestrated epithelia such as the choroid plexus and dura. In still further aspects, this reframes the perivascular niche as an active compartment connected to cerebrospinal fluid flow, not a passive byproduct of barrier leakage.

In various further embodiments, the invention can involve macrophage replacement at CNS borders. In further aspects, border-associated macrophages (BAMs) are yolk sac-derived and normally tolerogenic. In still further aspects, during chronic inflammation, BAMs can be progressively replaced by CCR2+ monocyte-derived macrophages (MDMs). Unlike BAMs, MDMs present antigen in a pro-inflammatory context and drive accumulation of pathogenic B and T cells in perivascular spaces. Together, these aspects can identify the perivascular space as a critical therapeutic target in progressive multiple sclerosis. According to other aspects, the present invention can provide compositions, methods, and systems that disrupt this pathogenic niche by administering engineered T cells specific for CLL-1 to deplete CLL-1-expressing MDMs. In further aspects, various embodiments can include combination with non-myelosuppressive lymphodepleting agents (including anti-CD20 antibodies, cladribine, alemtuzumab, and mitoxantrone) to reduce pathogenic B cells. This combination can prevent displacement of tolerogenic BAMs, reduce perivascular lymphocyte accumulation, and/or alter the course of disease.

In various aspects, multiple sclerosis (MS) can be traditionally regarded as an autoimmune disorder initiated by peripheral adaptive immune cells. In the prevailing model, autoreactive T and B lymphocytes are generated in the periphery, traverse the blood-brain barrier (BBB), and initiate demyelination and axonal injury within the central nervous system (CNS). The BBB is a specialized vascular interface composed of endothelial cells joined by tight junctions, pericytes, and astrocytic endfeet, which together limit indiscriminate entry of circulating cells and solutes into the CNS parenchyma. This “outside-in” paradigm has informed therapeutic development for the past three decades. Since the first MS therapy approval in 1993, more than twenty agents have been licensed, the majority of which reduce relapses and new inflammatory lesion activity by modulating peripheral immune function. These drugs demonstrate that relapsing disease activity is highly dependent on peripheral immune cell activation. Despite this progress, clinical progression of disability remains a central unmet need.

In further aspects, patients continue to accumulate irreversible disability even when relapses and MRI activity are nearly eliminated by current therapies, including highly effective B-cell-depleting agents. Available agents for progressive MS provide only partial benefit and do not halt or reverse disease in patients with advanced progression. Progression begins early in the disease course, indicating that mechanisms beyond peripheral immune activation drive disease biology from the outset. In recognition of this limitation, a concept of “smoldering inflammation” has been advanced, described as ongoing inflammatory activity that occurs “behind an intact blood-brain barrier.” This construct has been used to explain progression despite control of overt relapses. However, the distinction between “acute inflammation” driven by peripheral immune cell entry and “smoldering inflammation” occurring independently behind the BBB does not adequately reconcile with the clinical and pathological continuum of MS. Rather, the introduction of two putatively separate and independent processes reflects an incomplete understanding of a single underlying pathogenic mechanism that drives both relapse activity and progression. Thus, while the autoimmune “outside-in” model explains relapses, and the “smoldering inflammation” framework attempts to explain progression, neither fully accounts for the unified biology of disease. This gap defines the need for alternative approaches that specifically address the compartmentalized processes within the CNS that sustain progression.

In still further aspects, the invention can involve Border-Associated Macrophages and Monocyte-Derived Macrophages. Without wishing to being bound by a particular theory, the inventor has further reduced to practice various embodiments that include, for example, macrophage populations at CNS borders undergo pathological replacement that sustains these lymphocyte-rich niches. In yet further aspects, border-associated macrophages (BAMs) are yolk sac-derived, long-lived, and self-renewing. Under homeostasis they act as tolerogenic sentinels, clearing debris and producing regulatory cytokines such as IL-10 and TGF-B. In even further aspects, during neuroinflammation, BAMs are activated by damage-associated molecular patterns (DAMPs) and pathogen-associated molecular patterns (PAMPs). They secrete TNF-α, IL-1B, IL-6, and CCL2, which recruits CCR2+ monocytes from the circulation. In still further aspects, tThese monocytes can enter through fenestrated sites and differentiate into monocyte-derived macrophages (MDMs) within CNS border stroma. In even further aspect, replacement can occur: MDMs progressively displace yolk sac-derived BAMs from their niches.

In further aspects, the invention can involve pathogenic remodeling. Unlike BAMs, MDMs present antigen in a pro-inflammatory context, with costimulation and cytokine secretion that drive the accumulation and activation of pathogenic lymphocytes. This BAM-to-MDM replacement can reframe perivascular spaces as active inflammatory compartments rather than passive byproducts of fluid leakage, sustaining lymphocyte-rich niches that fuel MS progression.

In further aspects, the invention can involve interstitial Fluid as the Compartment for Lymphocyte Accumulation. In still further aspects, and without being bound by a particular theory, the inventor has reduced to practice various embodiments, for example, wherein multiple sclerosis follows the anatomy of interstitial fluid (ISF) pathways rather than dysfunction of the blood-brain barrier. These disclosed embodiments can include perivascular and meningeal inflammation as consequences of ISF trafficking and not barrier leakage.

In some aspects, lymphocytes observed in perivascular cuffs in multiple sclerosis may not enter the brain by transmigration across the blood-brain barrier. Instead, they remain within interstitial fluid compartments at all times. Interstitial fluid surrounds lymphocytes in secondary lymphoid organs such as lymph nodes, and the same principle applies within the central nervous system. Once lymphocytes enter the CNS through fenestrated endothelium at the blood-CS barrier (including the choroid plexus and circumventricular organs), they are carried into cerebrospinal fluid and subsequently distributed into interstitial fluid compartments of the brain.

In further aspects, these interstitial fluid compartments can include the perivascular (Virchow-Robin) spaces around veins, the subpial compartment beneath the glia limitans, the periependymal zone along the ventricles, and stromal spaces of circumventricular organs. In the subpial compartment, persistent lymphocyte residence can explain the formation of tertiary lymphoid structures, widely regarded as a pathologic hallmark of progressive disease but previous y unexplained. In the deep white matter, perivascular spaces along penetrating venules (Virchow-Robin extensions) and smaller interstitial clefts along myelinated axon bundles can likewise be filled with ISF. These spaces can be continuous with the parenchymal extracellular space (ECS) and participate directly in interstitial clearance. Their enlargement can be readily observed by MRI in aging, multiple sclerosis, and hypertension. Perivascular lymphocyte cuffing, a histopathological hallmark of MS, can therefore be explained by lymphocyte residence in IS pathways that anatomically surround penetrating venules and extend through white matter tracts. Thus, according the various embodiments, MS pathology can comprise lesions consistently appear in locations defined by interstitial fluid compartments, including periventricular Dawson's fingers, subpial demyelination with tertiary lymphoid structures, perivascular cuffs, deep white matter Virchow-Robin spaces, and the ependymal dot-dash sign on MRI, which can confirm that the disease follows IS anatomy rather than blood-brain barrier disruption.

Accordingly, the various aspects of the present disclosure can provide the biological foundation for the present invention: therapeutic compositions, methods, and systems that target perivascular niches in multiple sclerosis.

In one aspect, the invention provides engineered T cells specific for CLL-1 to selectively deplete CLL-1-expressing MDMs, in combination with non-myelosuppressive lymphodepleting agents (including anti-CD20 monoclonal antibodies, cladribine, alemtuzumab, and mitoxantrone) to reduce pathogenic B cells in IS compartments. In further aspects, this disclosed approaches can prevent pathological replacement of BAMs, disrupt the perivascular lymphocyte niche, and/or alter the course of disease.

The description provided herein is intended to support the enablement and written description requirements of the present claims and should not be interpreted as limiting the scope of the invention to any particular theory of disease mechanism.

In further aspects, in chronic multiple sclerosis, meningeal and perivascular spaces can develop tertiary lymphoid follicles. These structures can resemble miniature lymph nodes that arise in the wrong place. Within them, several immune populations can converge and interact in a way that sustains disease. In still further aspects, border-associated macrophages (BAMs), as used herein, may refer to the “good” resident macrophages, present from early development. Under normal conditions, they help maintain balance at CNS barriers. In yet further aspects, monocyte-derived macrophages (MDMs) can, over time, be circulating CCR2* monocytes to infiltrate the CNS and replace BAMs. These “bad” macrophages can occupy stromal niches, provide constant stimulation to lymphocytes, and can be the main drivers of chronic follicle activity. MDMs can express CLL-1, which marks them as distinct from the protective BAMs. B cells: Inside the follicle, B cells can collect and expand. Supported by the MDMs, they persist in a chronic, activated state and provide continuous antigenic drive. Killer T cells (CD8* cytotoxic T cells): These are the “good” lymphocytes, capable of controlling disease by eliminating chronically stimulated B cells. However, within the follicle they are held back. MDMs can suppress their activity, and over time the killer T cells become “exhausted”-they lose their ability to attack.

In even further aspects, this simple arrangement can explain the course of MS: In early disease, killer T cells are still active. Their bursts of activity cause relapses, which can be understood as attempts by the immune system to clear pathogenic niches. In advanced disease, killer T cells can be exhausted and no longer function. Relapses fade, but the follicles remain, sustained by MDMs and B cells. This state can drive steady progression of disability despite the absence of acute attacks. Thus, tertiary lymphoid follicles may not be bystanders but the engines of progression. Their persistence can reflect the replacement of protective BAMs with pathogenic MDMs, the unchecked survival of B cells, and the suppression and exhaustion of cytotoxic T cells.

Thus, in accordance with various aspects of the present disclosure, Stepwise Model of Follicle-Driven MS can comprise:
1. Early stage (relapsing MS):
   BAMs (resident “good” macrophages) still present at CNS borders.
   CCR2* monocytes begin to infiltrate and differentiate into MDMs (“bad” macrophages).
   Follicles start to form with clusters of B cells and infiltrating T cells.
   Killer T cells (CD8*) remain functional and attempt to attack B cells.
   Bursts of killer T-cell activity cause relapses→acute inflammatory episodes.
2. Intermediate stage (transition):
   MDMs progressively replace BAMs in stromal niches.
   MDMs provide constant stimulation that sustains follicle growth.
   Killer T cells are increasingly suppressed by inhibitory signals from MDMs.
   B cells expand and persist inside the follicle.
3. Chronic stage (progressive MS):
   BAMs largely displaced; follicles dominated by MDMs and B cells.
   Killer T cells become exhausted-they no longer mount effective attacks.
   Relapses decline or disappear, not because disease is controlled, but because the “good” killer T cells are worn out.
   Follicles persist and drive slow, steady progression of disability.

In further aspect, this model can highlight:
Relapses=Darwinian attempts by killer T cells to clear follicles.
Progression=state of follicle persistence after killer T-cell exhaustion.
The critical switch is the replacement of BAMs by CCR2*, CLL-1* MDMs, which both sustain follicles and hold killer T cells in check.

In accordance with the various aspects of the present disclosure, calprotectin is a heterodimer of the calcium-binding proteins S100A8 and S100A9 (historically referred to as “100A8/89”). It is a prominent damage-associated molecular pattern (DAMP) released by myeloid cells. Source in the CNS can include:
Border-associated macrophages (BAMs) are a key physiologic source of calprotectin at CNS interfaces, including meninges and perivascular spaces.
BAMs release calprotectin during phagocytosis of debris, aged erythrocytes, or microbial products, as part of their normal barrier-surveillance role.
Release is triggered by pattern recognition receptor (PRR) activation (e.g., TLR2, TLR4 engagement) and by local inflammatory stress.

Functional role:
In homeostasis, calprotectin contributes to innate immune surveillance by binding microbial products, sequestering divalent metal ions, and shaping the local cytokine milieu.
In disease, elevated calprotectin reflects activation of BAMs and related myeloid populations, serving as a measurable biomarker of compartmentalized inflammation.
In CSF, increased calprotectin levels have been associated with progressive MS and correlate with the expansion of pathogenic stromal macrophages that sustain tertiary lymphold structures.

Good vs. bad macrophage dichotomy:
Good BAMs: release calprotectin in a regulated fashion as part of physiologic immune surveillance.
Bad macrophages (CCR2*, CLL-1* MDMs): sustain chronic release in an unregulated, pro-inflammatory manner, linking elevated CS calprotectin to chronic follicle activity and progression.

According to various embodiments of the present disclosure, the therapeutic rationale can provide restoration of effective cytotoxic T-cell activity within the CNS by selectively depleting pathogenic myeloid populations. In progressive MS, border-associated macrophages (BAMs) that normally support CNS barrier function are progressively displaced by CCR2* monocyte-derived macrophages (MDMs). These infiltrating myeloid cells act as myeloid-derived suppressor cells, sustaining tertiary lymphoid structures (TLS) and delivering inhibitory signals that suppress CD8* cytotoxic T cells. Therefore, CD8+ T cells-which are protective in this context-become exhausted and unable to eliminate chronically activated B cells or dismantle TLS. Early in disease, bursts of CD8* activity manifest as relapses, but over time exhaustion leads to disappearance of relapses and steady progression.

In further aspects, various embodiments of the present invention can employ anti-CLL-1 CAR-T cells, engineered to recognize and deplete CLL-1* MDMs and stromal macrophages. In further aspects, this targeted depletion can include one or more of:
Removes the TLS-supporting myeloid scaffold, disrupting the persistence of pathogenic lymphoid aggregates.
Lifts suppressive signaling on CD8* T cells, restoring their effector capacity.
Reactivates endogenous immune clearance, enabling cytotoxic T cells to resume their physiologic role in controlling chronic immune niches.

Thus, according to some aspects of the invention, anti-CLL-1 CAR-T cell therapy in MS may not suppress immunity broadly, but instead removes the pathogenic myeloid populations that restrain protective cytotoxic T cells. By eliminating CLL-1* macrophages, the therapy can reactivate a beneficial immune program and provides a strategy to halt disease progression.

In various aspects, autoimmune disorders can be diseases where a body's immune system attacks self-antigens. Attempts to treat autoimmune disorders have met with limited success. This is due, in part, to the fact that the etiology of autoimmune disorders is a complex response based in part on a combination of factors, including, without limitation, genetic make-up of individual, gender or hormonal status, bacterial or viral infection, metal or chemical toxin exposure, vaccinations or immunizations, stress, trauma, smoking and/or nutritional deficiencies.

Therefore, compounds, compositions, and methods that can reduce a symptom associated with an autoimmune disorder, inflammation associated with an autoimmune disorder, and/or a transplant rejection would be highly desirable.

In MS, particularly in progressive disease, perivascular stromal macrophages of monocyte-derived origin can establish sustained interactions with infiltrating T lymphocytes. These macrophages, which replace the homeostatic yolk sac-derived border-associated macrophages, can upregulate CLL-1 (CLEC12A) along with high levels of MHC-II and co-stimulatory ligands, while also expressing inhibitory checkpoint ligands such as PD-L1, B7 family members, and related molecules. The result is a pathological “handshake” with perivascular T cells: autoreactive T cells receive sufficient stimulation to persist and secrete pro-inflammatory cytokines, yet are simultaneously restrained in an exhausted or partially dysfunctional state. This interaction drives the chronic, smoldering inflammation characteristic of progressive MS, including cytokine-mediated axonal injury, demyelination, and neurodegeneration. Therapeutic depletion of the CLL-1-positive macrophage subset is therefore expected to disrupt these maladaptive macrophage-T cell interactions, thereby reducing perivascular lymphocyte cuffing, restoring homeostatic border macrophage populations, and altering the trajectory from relapsing to progressive MS.

In further aspects, border-associated macrophages (BAMs) are a specialized subset of tissue-resident macrophages located along the interfaces of the central nervous system (CNS), particularly at the borders of the brain, such as the meninges, the perivascular spaces, and the choroid plexus. These cells play a critical role in maintaining CNS homeostasis, mediating immune surveillance, and responding to injury or infection. In the context of multiple sclerosis (MS), BAMs contribute to neuroinflammation and have been implicated in the progression of demyelinating lesions. BAMs, including choroid plexus macrophages (CPMs), express various surface markers, including CLL-1 (CLEC12A), making them a target for immunotherapeutic interventions such as CAR-T cell therapy or monoclonal antibodies. Targeting CLL-1 (CLEC12A)-expressing BAMs can modulate the neuroinflammatory environment in MS, potentially reducing inflammation and halting disease progression. As used herein, ‘border-associated macrophages’ (BAMs) refers broadly to macrophages at the choroid plexus, meninges, and perivascular spaces. In multiple sclerosis and related conditions, these compartments become populated by monocyte-derived, CLL-1-positive macrophages that replace the original yolk sac-derived BAMs. Such infiltrating cells may be referred to as ‘BAM-like’ or ‘disease-associated BAMs,’ and these are the intended targets of the present invention.

Resident and Non-Resident Monocytes: Monocytes, derived from different developmental origins, are a crucial component of the innate immune system and are categorized into resident and non-resident populations. Resident monocytes, often derived from yolk sac progenitors during embryonic development, migrate early into tissues where they remain for long periods, contributing to tissue homeostasis, repair, and immune regulation. These cells establish a local immune environment and can differentiate into tissue-resident macrophages or dendritic cells. Their early lineage, tied to the yolk sac, grants them a role as the silent sentinels of tissue integrity, maintaining order and responding to subtle changes in tissue health.

On the other hand, non-resident monocytes, which are continuously replenished from bone marrow-derived hematopoietic stem cells (HSCs) throughout life, circulate in the blood and are rapidly recruited to sites of inflammation, infection, or injury. These cells, often referred to as inflammatory monocytes, act as first responders, migrating to affected areas and differentiating into macrophages or dendritic cells to mediate the immune response.

In MS, pathogenic immune cells are believed to traffic into the central nervous system not primarily by breaching the blood-brain barrier from within, but via the choroid plexus, where the fenestrated endothelium overlies a stromal compartment that facilitates entry. This route allows both autoreactive T cells and monocyte-derived macrophages to access the CNS border regions. In MS, these infiltrating monocyte-derived cells replace the original yolk sac-derived border-associated macrophages, giving rise to CLL-1-positive disease-associated BAM-like cells. The fenestrated architecture of the choroid plexus thus provides a rational and feasible point of access for CLL-1-directed CAR-T cells to reach their intended targets in vivo.

Unlike their tissue-resident counterparts, non-resident monocytes reflect the dynamic nature of immune surveillance, constantly patrolling the vascular highways in readiness to combat threats. In the context of multiple sclerosis (MS), both resident and non-resident monocytes contribute to the chronic neuroinflammation that drives disease progression. Targeting monocytes, particularly those expressing surface markers such as CLL-1 (CLEC12A), offers a therapeutic approach to modulate the inflammatory response in MS, potentially preventing the migration and differentiation of these cells into neuroinflammatory sites within the central nervous system.

In various aspects, the presently disclosed subject matter provides methods and compositions for treating CLL-1 (CLEC12A)-expressing myeloid cell-mediated autoimmune diseases and disorder, such as multiple sclerosis, by targeting C-type lectin-like molecule-1 (CLL-1 or CLEC12A) expressed on border-associated macrophages (BAMs) and other immune cells involved in neuroinflammation.

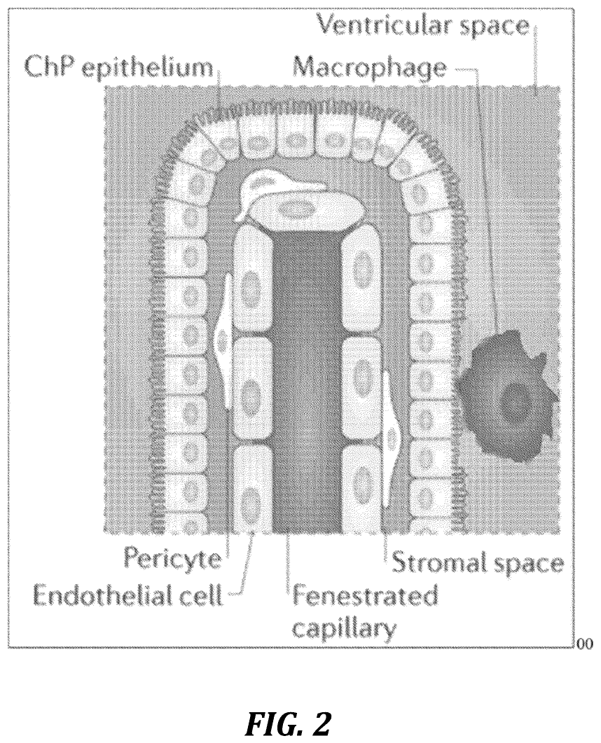

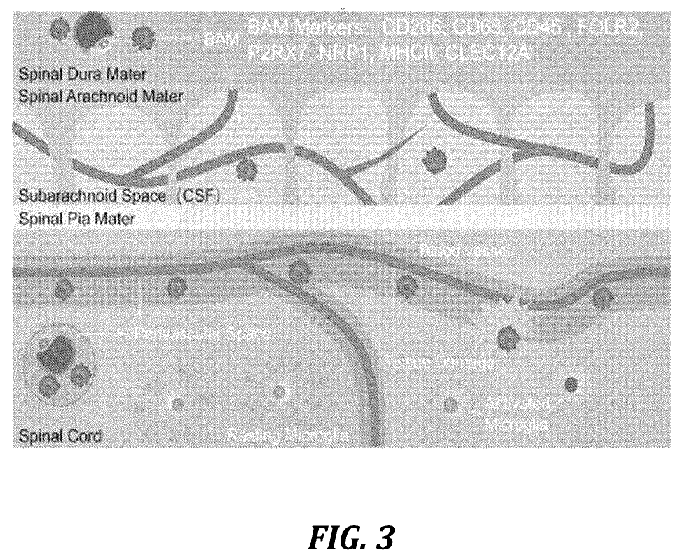

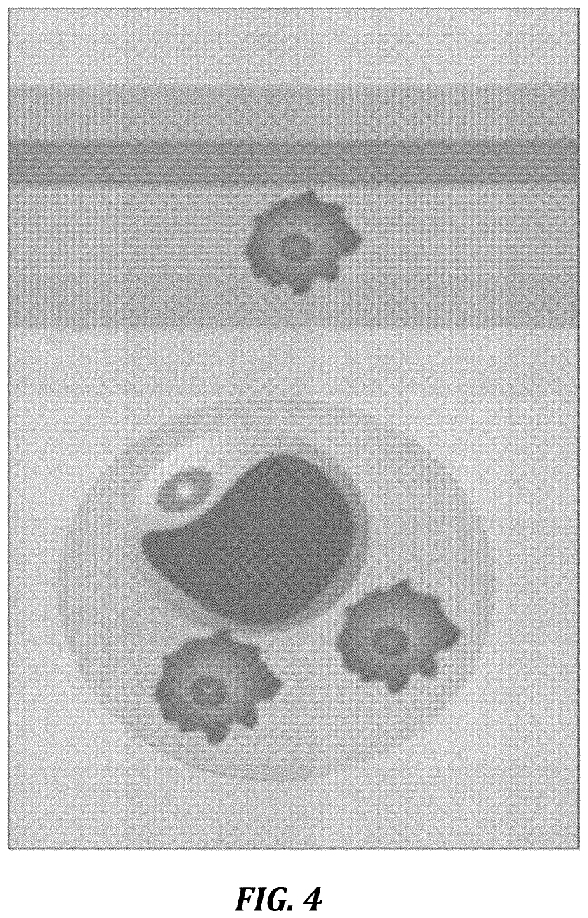

In contrast to conventional models that treat inflammation and neurodegeneration in multiple sclerosis as separate processes, the present disclosure, in some aspects, describes a unified mechanism. In this view, monocyte-derived, CLL-1-positive macrophages can infiltrate the CNS through fenestrated regions of the choroid plexus and other border sites. These cells can both initiate relapsing inflammatory episodes and, through expression of inhibitory ligands such as PD-L1, CD80, and CD86, suppress T-cell activity over time. This dual role can explain the clinical transition from relapsing to progressive disease and supports the inventive strategy of selectively depleting such macrophages with CLL-1-directed CAR-T cells. FIG. 2 depicts a cross-sectional view of the choroid plexus and its fenestrated capillary structure. The configuration allows administered engineered T cells to access the central nervous system through the choroid plexus, and to interact with CNS-resident myeloid cells. Such trafficking and access routes are relevant to aspects of the invention that target compartmentalized neuroinflammation via depletion or modulation of myeloid cells within CNS compartments. FIG. 3 shows the anatomical distribution of border-associated macrophages (BAMs) along the spinal cord and meningeal spaces. BAMs are implicated in the maintenance of inflammatory environments in the CNS. The disclosed CAR-T cells are designed to recognize and eliminate these CLL-1-expressing cells, reducing chronic neuroinflammation. FIG. 4 provides a higher magnification of border-associated macrophages located perivascularly within regions behind the blood-brain barrier, including both acute and chronic lesions. The inventive compositions selectively deplete or modulate these perivascular macrophage populations, which contribute to disease progression and compartmentalized CNS inflammation in multiple sclerosis.

To this end, multiple sclerosis (MS) is an autoimmune disorder characterized by demyelination and neurodegeneration in the central nervous system (CNS). Current understanding of MS pathophysiology indicates the involvement of two largely independent but concurrent inflammatory pathways that drive disease progression from the onset: acute neuroinflammation and smoldering neuroinflammation.

Acute neuroinflammation can be primarily driven by peripheral autoreactive T and B cells that cross the blood-brain barrier (BBB) and cause inflammatory demyelination. Most current MS therapies target this pathway effectively, resulting in significant reduction of relapses in relapsing-remitting MS (RRMS). Smoldering neuroinflammation, on the other hand, is mediated by CNS-resident immune cells, including border-associated macrophages (BAMs), that promote ongoing neurodegeneration behind an intact BBB. This pathway is not effectively addressed by current therapies and contributes to disease progression, particularly in secondary progressive MS (SPMS) and primary progressive MS (PPMS).

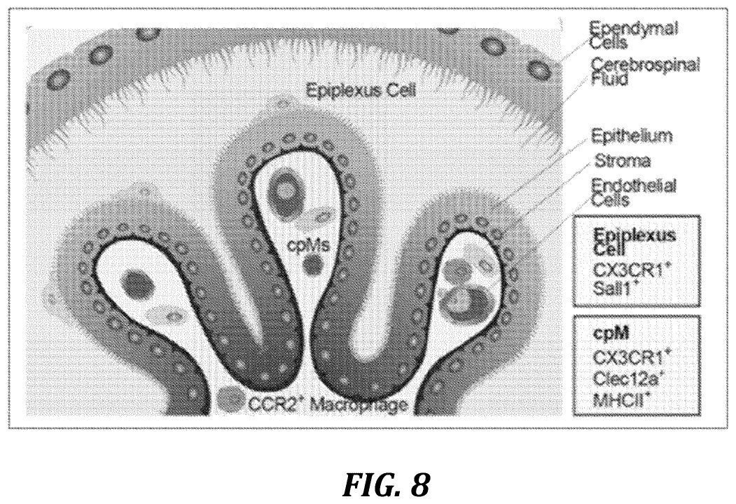

In further aspects, BAMs can represent a specialized subset of tissue-resident macrophages located at the interfaces between the CNS and the periphery, including the choroid plexus, meninges, and perivascular spaces (FIGS. 3 and 4). These macrophages express various markers including CD206, CD63, CD45, FOLR2, P2RX7, NRP1, MHC II, and importantly, CLL-1 (also known as CLEC12A) (FIG. 8). BAMs play crucial roles in CNS immune surveillance, phagocytosis of debris, antigen presentation, and regulating the exchange of cells and cytokines between the CNS and bloodstream.

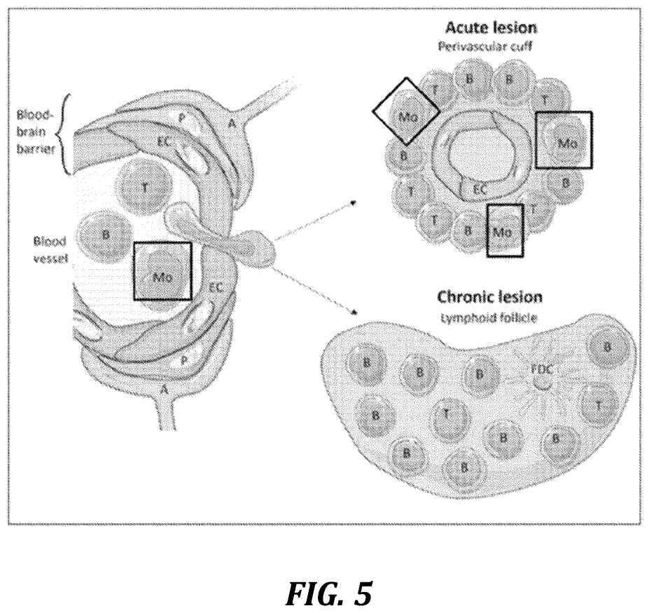

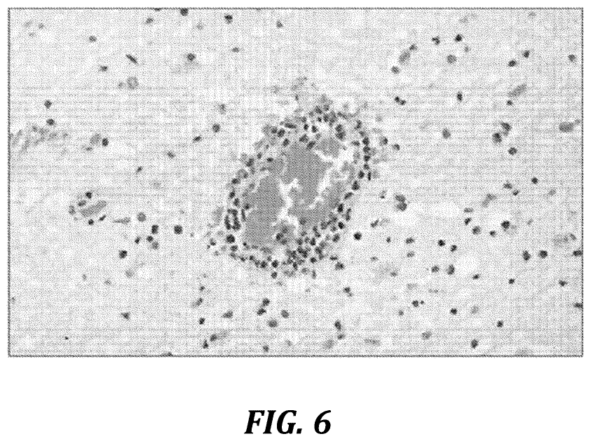

FIG. 5 illustrates the presence of perivascular lymphocyte cuffing, a recognized pathological hallmark of multiple sclerosis. BAMs reside within these perivascular cuffs and contribute to neuroinflammation. Therapeutic intervention targeting CLL-1-expressing cells as disclosed herein may reduce or prevent accumulation of lymphocytes and pathologic BAM activity in these regions. FIG. 6 demonstrates histological findings of perivascular lymphocyte accumulation (cuffing) in CNS tissues from subjects with multiple sclerosis. This figure provides experimental context relevant to the assessment of treatment efficacy for the disclosed methods and compositions, based on modulation of perivascular immune cell populations.

In MS, BAMs can contribute to pathological inflammation through various mechanisms. The choroid plexus, where BAMs reside, may serve as an entry point for autoreactive lymphocytes before BBB disruption, contributing to CNS infiltration and disease progression (FIG. 2). The blood-cerebrospinal fluid barrier (BCSFB) at the choroid plexus has differential tight junctions compared to the BBB, making it more permeable to certain cells and molecules.

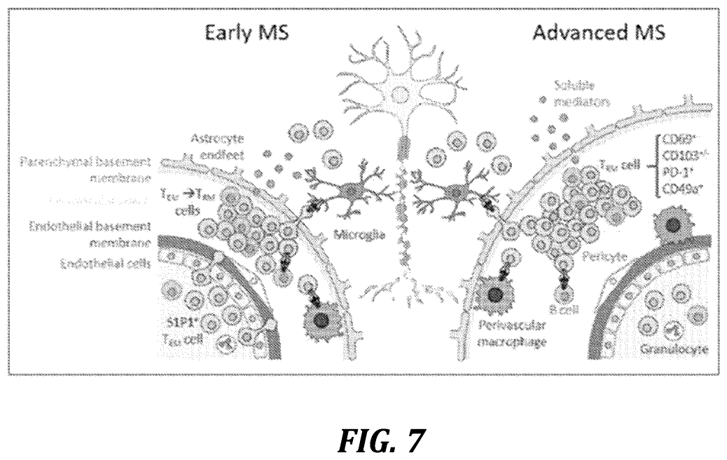

Systemic inflammation can impair tight junctions at the BCSFB, increasing immune cell trafficking at this barrier. This process is regulated by IFNγ-dependent activation, with chemokines like CCL20 and CX3CL1 guiding CCR6+ and CX3CR1+ leukocytes to the choroid plexus. Systemic inflammation upregulates CXCLI, CCL2, MMP8, and MMP9, further impairing tight junctions and increasing immune cell trafficking. Choroid plexus macrophages (CPMs) in MS can express high levels of antigen-presentation molecules (MHC II) and produce pro-inflammatory cytokines like IL-1B. These cells express CLL-1 and contribute significantly to systemic inflammation in MS. The perivascular accumulation of immune cells, including BAMs, forms perivascular cuffs, which are hallmark pathological findings in MS (FIGS. 5 and 6). As presented herein, the perivascular cuffs observed in MS represent downstream accumulation sites within an interstitial fluid continuum, and that the true entry point for pathogenic and therapeutic T cells is the fenestrated endothelium of the choroid plexus stroma, which is contiguous with these perivascular space. FIG. 7 compares pathological changes in CNS tissues at early and advanced stages of multiple sclerosis, with an emphasis on the accumulation and function of perivascular macrophages. The inventive treatment methods aim to interfere with the sustained inflammatory activity driven by these myeloid populations in progressive disease. FIG. 8 shows border-associated macrophages located in the choroid plexus, demonstrating CLL-1 expression that provides a rational target for CAR-T cell mediated therapy. These anatomical features highlight the central role of perivascular and choroid plexus macrophages in sustaining compartmentalized CNS inflammation.

Smoldering neuroinflammation has not been directly addressed by current MS therapies due to the inability of most drugs to cross the BBB at pharmacologically relevant levels. However, therapeutic agents capable of crossing the BCSFB and targeting CLL-1-expressing BAMs may provide a novel approach to address this unmet medical need. Furthermore, it has previously been suggested that multiple sclerosis involves two largely independent processes: (i) relapsing inflammatory activity and (ii) smoldering neurodegeneration occurring behind an intact blood-brain barrier. In contrast, as presented herein, pathological observations such as perivascular lymphocyte cuffing are more consistent with a unified process in which immune cells enter via fenestrated and stromal regions of the choroid plexus and other CNS borders, rather than traversing an intact barrier from within. Pathogenic myeloid and lymphoid cells converge at border-associated sites, where yolk sac-derived macrophages are replaced by monocyte-derived, CLL-1-positive macrophages. Targeting these disease-associated macrophages with CLL-1-directed CAR-T cells is distinct from prior work in hematologic malignancy (e.g., AML) and from existing immune-targeting strategies in MS (e.g., anti-CD19 CAR-T).

In further aspects, C-type lectin-like molecule-1 (CLL-1, also known as CLEC12A) is a type II transmembrane glycoprotein belonging to the C-type lectin/C-type lectin-like domain (CTL/CTLD) family. CLL-1 is expressed on various myeloid cells, including dendritic cells, monocytes, granulocytes, and importantly, border-associated macrophages (BAMs) in the CNS.

As disclosed herein, the present disclosure provides various embodiments wherein CLL-1 expression on BAMs can be a target for therapeutic intervention in MS. By targeting CLL-1, the disclosed therapies can selectively deplete pathogenic BAMs that contribute to smoldering neuroinflammation while sparing other immune cells necessary for normal immune function. Importantly, CLL-1 expression is upregulated on disease-associated macrophages in multiple sclerosis relative to yolk sac-derived resident BAMs in the healthy CNS. This differential expression provides a therapeutic window, enabling selective depletion of pathogenic, monocyte-derived CLL-1-positive macrophages while sparing a substantial fraction of protective resident macrophages.

### Compositions

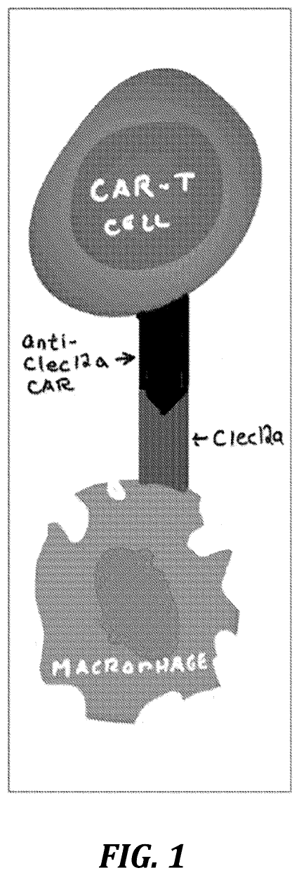

Therefore, disclosed are compositions containing one or more agents that directly target and deplete CLL-1-expressing myeloid cells via the binding of the CLL-1 receptor. This interaction and subsequent depletion prevents pathological inflammation mediated by these immune cells. CLL-1 may be targeted using various therapeutic modalities, including chimeric antigen receptor (CAR)-T cell therapy, monoclonal antibodies, bi-specific antibodies, and antibody-drug conjugates. FIG. 1 illustrates an engineered T cell expressing a chimeric antigen receptor (CAR) targeting C-type lectin-like molecule-1 (CLL-1, also known as CLEC12A), binding to a CLL-1-expressing myeloid cell. Aspects of the invention provide for administration of such engineered T cells, including CAR-T cells, to selectively target and modulate CLL-1-expressing border-associated macrophages (BAMs) implicated in the pathogenesis of multiple sclerosis and related autoimmune or inflammatory conditions.

### A. Car-Cells

In some aspects, the composition includes a cell including a chimeric antigen receptor (CAR), for example a chimeric antigen receptor (CAR)-T cell. The cell can comprise a white blood cell, including a lymphocyte. In some embodiments, the lymphocyte can comprise a T-lymphocyte. In some embodiments, the composition can comprise one or more of the following T cell types: CD4+ T cells, CD8+ T cells, gamma delta (γδ) T cells, memory T cells, naive T cells, exhausted T cells, T regulatory cells (Tregs), natural killer T cells (NKT cells), invariant natural killer T cells (INKT cells), mucosal associated invariant T cells (MAIT cells), effector T cells, stem cell memory T cells (Tscm), double-negative T cells (DN T cells). Upon genetic modification, the T-lymphocyte expresses the CAR and becomes a CAR-T lymphocyte. Other modifications or mutations, either intentional or unintentional, can also cause changes in the T-lymphocyte. In some embodiments, the administration of the CAR-T-lymphocyte causes elimination of at least an amount of monocytes. In some embodiments, the monocytes are endogenous. In some embodiments, the CAR-T lymphocyte is administered to a monocyte. In this application, T-lymphocyte and T-cell are used interchangeably.

In some embodiments, a CAR-T cell, a genetically modified T-cell, is administered intravenously for the purpose of treating the autoimmune disorder. Subsequent to being administered, the CAR-T cell travels through a blood vessel to a capillary. The CAR-T enters the interstitial fluid (ISF), where the CAR-T cell engages the monocyte. (In this application, the ISF can be used interchangeably with the term “stroma.”) The monocyte is detected based on an expression of a receptor on a surface of the monocyte that binds with a CAR on a surface of the CAR-T cell. In some embodiments, the receptor on the monocyte comprises a C-type lectin/C-type lectin-like domain (CTL/CTLD). In some embodiments, the receptor on the monocyte is CLL-1 or CLECI2A.

In some embodiments, the administered CAR-T cells enter the central nervous system by trafficking through fenestrated endothelial vessels of the choroid plexus into the underlying stromal compartment. This compartment contains interstitial fluid that is continuous with perivascular fluid spaces throughout the CNS parenchyma. Accordingly, CAR-T cells administered to a subject with multiple sclerosis may distribute via this interstitial continuum, thereby reaching perivascular cuffs and other sites of compartmentalized inflammation where CLL-1-expressing myeloid cells accumulate.

In some embodiments, engineered T cells are modified to reduce or eliminate inhibitory signaling mediated by checkpoint receptor-ligand interactions. Such modifications may include disruption of checkpoint receptors (e.g., PD-1, TIM-3, CTLA-4, LAG-3, TIGIT) and/or alterations conferring resistance to inhibitory ligands, such as members of the B7 family (e.g., CD80, CD86, PD-L1, PD-L2). These modifications may enhance the persistence and efficacy of CLL-1-directed CAR-T cells in treating MS.

In some embodiments, the monocyte is a resident immune cell, developmentally native to an area where it is detected by the CAR-T cell. In some embodiments, the resident immune cell stays in a specific tissue or organ for a long time, rather than circulating throughout the body. These cells “take up residence” in a certain area, like the skin, lungs, or brain, and are ready to act quickly if there's an infection or injury in that specific location.

Unlike other immune cells that travel through the bloodstream, resident immune cells can be specialized to monitor and protect the tissue where they live. In some embodiments, in order to treat the autoimmune disorder, the CAR-T cells are targeted towards elimination of the resident immune cells. In some embodiments, the monocyte can be a non-resident immune cell that has migrated to or been recruited to the area where it is detected by the CAR-T cell. The non-resident immune cell can also be eliminated by the CAR-T cell in order to treat a disorder, including an autoimmune disorder. Upon elimination of the resident immune cells, risk of systemic inflammation impairing tight junctions of the overlying epithelial cells can decrease, thus decreasing immune cell trafficking at the blood-CSF barrier.

In further aspects, CAR-T cell therapy can offer several advantages, including high specificity, potent cytotoxicity, and potential for long-term persistence and surveillance. In further aspects, anti-CLL-1 CAR-T cells may be particularly effective in treating MS by targeting CLL-1-expressing BAMs that contribute to smoldering neuroinflammation (FIG. 1). These cells may cross the BCSFB more readily than the BBB, allowing them to access BAMs in the choroid plexus and other CNS border regions.

In still further aspects, chimeric antigen receptor (CAR)-T cell therapy can involve genetically modifying T cells to express a synthetic receptor that recognizes a specific target antigen. In some embodiments, the disclosed compositions can include engineered CAR-T cells expressing a CAR that targets CLL-1, designated as CB-012.

In yet further aspects, the anti-CLL-1 CAR may comprise an extracellular domain containing an anti-CLL-1 binding domain, a transmembrane domain, and/or an intracellular signaling domain. The anti-CLL-1 binding domain may be derived from an anti-CLL-1 antibody or other CLL-1-binding moiety. The transmembrane domain may connect the extracellular and intracellular components. The intracellular domain may contain signaling components that activate T cell effector functions upon CAR engagement with CLL-1, including a CD3ζ signaling domain and one or more costimulatory domains such as CD28, 4-1BB, or OX40.

In other embodiments, the CAR-T cells may comprise one or more genetic modifications to enhance their therapeutic efficacy, persistence, and safety. These modifications may include one or more of:
T Cell Receptor (TCR) Knockout: Elimination of the endogenous TCR to prevent graft-versus-host disease and improve CAR-T cell specificity;
Programmed Cell Death Protein 1 (PD-1) Knockout: Removal of the PD-1 immune checkpoint receptor to prevent T cell exhaustion, which may be particularly important in MS where PD-1 is elevated on T cells;
Beta-2-Microglobulin (B2M) Knockout: Elimination of B2M to prevent expression of classical MHC class I molecules, reducing recognition by host cytotoxic T cells;
B2M-HLA-E Insertion: Addition of B2M fused to HLA-E to prevent natural killer (NK) cell-mediated killing while maintaining the benefit of B2M knockout;
In further aspects, these genetic modifications can work synergistically to create a “cloaked and armored” CAR-T cell that can effectively target CLL-1-expressing BAMs while evading host immune clearance mechanisms, potentially leading to enhanced persistence and efficacy. In still further aspects, the CAR-T cells may be either autologous (derived from the patient's own T cells) or allogeneic (derived from a healthy donor). Allogeneic CAR-T cells may offer advantages in terms of availability, standardization, and quality control, but may require additional genetic modifications to prevent immune rejection and graft-versus-host disease. In yet further aspects, other therapeutic modalities may also be used to target CLL-1-expressing BAMs in MS. These additional modalities may be used alone or in combination with CAR-T cell therapy, depending on the specific clinical context and patient characteristics.

### B. Monoclonal Antibodies

In some aspects, the disclosed composition may include one or more anti-CLL-1 monoclonal antibodies. In further aspects, anti-CLL-1 monoclonal antibodies may bind to CLL-1 on BAMs and induce cell death through various mechanisms, including antibody-dependent cellular cytotoxicity (ADCC), complement-dependent cytotoxicity (CDC), and/or direct inhibition of CLL-1 signaling. Monoclonal antibodies may offer advantages in terms of manufacturing, dosing flexibility, and safety profile, but may have lower potency compared to CAR-T cells.

In some embodiments, the disclosure provides monoclonal antibodies (mAbs) that specifically bind to CLL-1 (CLEC12A) on pathogenic immune cells, including dendritic cells and monocytes, for the treatment of MS at different stages. In some embodiments, monoclonal antibodies are administered to patients with Relapsing-Remitting MS (RRMS) to reduce relapse frequency by depleting CLL-1 (CLEC12A)-expressing immune cells responsible for triggering autoimmune attacks. In other embodiments, monoclonal antibodies are administered to patients with Secondary Progressive MS (SPMS) to reduce neuroinflammation and slow progression. In still other embodiments, monoclonal antibodies are administered to patients with Primary Progressive MS (PPMS) to reduce neuroinflammation and slow neurological decline by targeting CLL-1 (CLEC12A)-expressing cells. In other embodiments, the anti-CLL-1 AmAbs may function via antibody-dependent cellular cytotoxicity (ADCC), complement-dependent cytotoxicity (CDC), or direct inhibition of CLL-1 (CLEC12A)-mediated pathways involved in immune cell activation.

### C. Bi-Specific Antibodies

In some aspects, the disclosed composition may include one or more Bi-specific antibodies. In further aspects, the Bi-specific antibodies target CLL-1 and another immune receptor (e.g., CD3 on T cells) and can redirect endogenous immune cells to eliminate CLL-1-expressing BAMs. These antibodies can provide a bridge between effector cells and target cells, enhancing immune-mediated cytotoxicity without the need for genetic modification of T cells.

### D. Antibody-Drug Conjugates (Adcs)

In some aspects, the disclosed composition may include one or more antibody-drug conjugates. In further aspects, ADCs include anti-CLL-1 antibodies conjugated to cytotoxic agents that are released upon internalization of the antibody-antigen complex. ADCs may deliver potent cytotoxic agents specifically to CLL-1-expressing cells while minimizing systemic toxicity.

As discussed herein, these alternative therapeutic modalities may be considered based on various factors, including the specific clinical presentation, disease stage, comorbidities, and patient preferences. They may also be used sequentially or in combination with CAR-T cell therapy to enhance efficacy or address treatment resistance.

### Formulations

The disclosed compositions can be formulated in a pharmaceutical composition. Pharmaceutical compositions including antigens, adjuvants, and the combination thereof are provided. Pharmaceutical compositions can be for administration by parenteral (intramuscular, intraperitoneal, intravenous (IV) or subcutaneous injection), enteral, transdermal (either passively or using iontophoresis or electroporation), or transmucosal (nasal, pulmonary, vaginal, rectal, or sublingual) routes of administration or using bioerodible inserts and can be formulated in dosage forms appropriate for each route of administration. In some embodiments, the compositions are administered systemically, for example, by intravenous, intrathecal, or intraperitoneal administration, in an amount effective for delivery of the compositions to targeted cells. Most typically, the compositions are administered by intramuscular, intradermal, subcutaneous injection or infusions, or intravenous injection or infusion, or by intranasal delivery.

### Parenteral Formulations

The compositions described herein can be formulated for parenteral administration. For example, parenteral administration may include administration to a patient intravenously, intradermally, intrathecally, intraperitoneally, intraventricular, intramuscularly, subcutaneously, by injection, by infusion, etc.

Parenteral formulations can be prepared as aqueous compositions using techniques is known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the active compounds as the free acid or base or pharmacologically acceptable salts thereof can be prepared in water or another solvent or dispersing medium suitably mixed with one or more pharmaceutically acceptable excipients including, but not limited to, surfactants, dispersants, emulsifiers, pH modifying agents, viscosity modifying agents, and combination thereof.

Suitable surfactants may be anionic, cationic, amphoteric or nonionic surface-active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-. beta.-alanine, sodium N-lauryl-. beta.-iminodipropionate, myristoamphoacetate, lauryl betaine and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s).

The formulation is typically buffered to a pH of 3-8 for parenteral administration upon reconstitution. Suitable buffers include, but are not limited to, phosphate buffers, acetate buffers, and citrate buffers.

Water-soluble polymers are often used in formulations for parenteral administration. Suitable water-soluble polymers include, but are not limited to, polyvinylpyrrolidone, dextran, carboxymethylcellulose, and polyethylene glycol.

Sterile injectable solutions can be prepared by incorporating the active compounds in the required amount in the appropriate solvent or dispersion medium with one or more of the excipients listed above, as required, followed by filtered sterilization. Generally, dispersions are prepared by incorporating the various sterilized active ingredients into a sterile vehicle which contains the basic dispersion medium and the required other ingredients from those listed above.

In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The powders can be prepared in such a manner that the particles are porous in nature, which can increase dissolution of the particles. Methods for making porous particles are well known in the art.

### Intrathecal Formulations

Suitable parenteral formulations for intrathecal administration can include solutions, suspensions, or dispersions of the engineered T cells or compositions thereof in a physiologically compatible carrier. Intrathecal formulations may comprise isotonic saline, artificial cerebrospinal fluid, Ringer's solution, or other pharmaceutically acceptable buffers, optionally supplemented with stabilizers, cryoprotectants, or excipients known in the art to preserve cell viability and function. Intrathecal administration may be achieved by lumbar puncture, intraventricular infusion, or catheter-based delivery, using methods well known in clinical neurology. In some embodiments, the intrathecal formulation comprises cryopreserved CAR-T cells thawed immediately prior to administration. In other embodiments, the intrathecal formulation may include co-administration of immunomodulatory agents, lymphodepleting agents, or checkpoint inhibitors to enhance efficacy. These approaches are compatible with routine clinical procedures for intrathecal delivery and are intended to encompass all pharmaceutically acceptable intrathecal routes of administration.

### Controlled Release Formulations

The parenteral formulations described herein can be formulated for controlled release including immediate release, delayed release, extended release, pulsatile release, and combinations thereof. For parenteral administration, the one or more compounds, and optional one or more additional active agents, can be incorporated into microparticles, nanoparticles, or combinations thereof that provide controlled release of the compounds and/or one or more additional active agents. In embodiments wherein the formulations contains two or more agents, the agents can be formulated for the same type of controlled release (e.g., delayed, extended, immediate, or pulsatile) or the agents can be independently formulated for different types of release (e.g., immediate and delayed, immediate and extended, delayed and extended, delayed and pulsatile, etc.).

For example, the compounds and/or one or more additional active agents can be incorporated into polymeric microparticles, which provide controlled release of the drug(s). Release of the agent(s) is controlled by diffusion of the agent(s) out of the microparticles and/or degradation of the polymeric particles by hydrolysis and/or enzymatic degradation. Suitable polymers include ethylcellulose and other natural or synthetic cellulose derivatives. Polymers, which are slowly soluble and form a gel in an aqueous environment, such as hydroxypropyl methylcellulose or polyethylene oxide, can also be suitable as materials for drug containing microparticles. Other polymers include, but are not limited to, polyanhydrides, poly(ester anhydrides), polyhydroxy acids, such as polylactide (PLA), polyglycolide (PGA), poly(lactide-co-glycolide) (PLGA), poly-3-hydroxybutyrate (PHB) and copolymers thereof, poly-4-hydroxybutyrate (P4HB) and copolymers thereof, polycaprolactone and copolymers thereof, and combinations thereof.

Alternatively, the agent(s) can be incorporated into microparticles prepared from materials which are insoluble in aqueous solution or slowly soluble in aqueous solution, but are capable of degrading within the GI tract by means including enzymatic degradation, surfactant action of bile acids, and/or mechanical erosion. As used herein, the term “slowly soluble in water” refers to materials that are not dissolved in water within a period of 30 minutes. Preferred examples include fats, fatty substances, waxes, wax-like substances and mixtures thereof. Suitable fats and fatty substances include fatty alcohols (such as lauryl, myristyl stearyl, cetyl or cetostearyl alcohol), fatty acids and derivatives, including but not limited to fatty acid esters, fatty acid glycerides (mono-, di- and tri-glycerides), and hydrogenated fats. Specific examples include, but are not limited to hydrogenated vegetable oil, hydrogenated cottonseed oil, hydrogenated castor oil, hydrogenated oils available under the trade name Sterotex®, stearic acid, cocoa butter, and stearyl alcohol. Suitable waxes and wax-like materials include natural or synthetic waxes, hydrocarbons, and normal waxes. Specific examples of waxes include beeswax, glycowax, castor wax, carnauba wax, paraffins and candelilla wax. As used herein, a wax-like material is defined as any material, which is normally solid at room temperature and has a melting point of from about 30° to 300° C.

In some cases, it may be desirable to alter the rate of water penetration into the microparticles. To this end, rate-controlling (wicking) agents can be formulated along with the fats or waxes listed above. Examples of rate-controlling materials include certain starch derivatives (e.g., waxy maltodextrin and drum dried corn starch), cellulose derivatives (e.g., hydroxypropylmethyl-cellulose, hydroxypropylcellulose, methylcellulose, and carboxymethyl-cellulose), alginic acid, lactose and talc. Additionally, a pharmaceutically acceptable surfactant (for example, lecithin) may be added to facilitate the degradation of such microparticles. Proteins, which are water insoluble, such as zein, can also be used as materials for the formation of agent containing microparticles. Additionally, proteins, polysaccharides and combinations thereof, which are water-soluble, can be formulated with agent into microparticles and subsequently cross-linked to form an insoluble network. For example, cyclodextrins can be complexed with individual agent molecules and subsequently cross-linked.

Encapsulation or incorporation of agent into carrier materials to produce agent-containing microparticles can be achieved through known pharmaceutical formulation techniques. In the case of formulation in fats, waxes or wax-like materials, the carrier material is typically heated above its melting temperature and the agent is added to form a mixture comprising agent particles suspended in the carrier material, agent dissolved in the carrier material, or a mixture thereof. Microparticles can be subsequently formulated through several methods including, but not limited to, the processes of congealing, extrusion, spray chilling or aqueous dispersion. In a preferred process, wax is heated above its melting temperature, agent is added, and the molten wax-agent mixture is congealed under constant stirring as the mixture cools. Alternatively, the molten wax-agent mixture can be extruded and spheronized to form pellets or beads. These processes are known in the art. For some carrier materials it may be desirable to use a solvent evaporation technique to produce agent-containing microparticles. In this case agent and carrier material are co-dissolved in a mutual solvent and microparticles can subsequently be produced by several techniques including, but not limited to, forming an emulsion in water or other appropriate media, spray drying or by evaporating off the solvent from the bulk solution and milling the resulting material.

In some embodiments, agent in a particulate form is homogeneously dispersed in a water-insoluble or slowly water soluble material. To minimize the size of the agent particles within the composition, the agent powder itself may be milled to generate fine particles prior to formulation. The process of jet milling, known in the pharmaceutical art, can be used for this purpose. In some embodiments drug in a particulate form is homogeneously dispersed in a wax or wax like substance by heating the wax or wax like substance above its melting point and adding the drug particles while stirring the mixture. In this case a pharmaceutically acceptable surfactant may be added to the mixture to facilitate the dispersion of the drug particles.

The particles can also be coated with one or more modified release coatings. Solid esters of fatty acids, which are hydrolyzed by lipases, can be spray coated onto microparticles or drug particles. Zein is an example of a naturally water-insoluble protein. It can be coated onto drug containing microparticles or drug particles by spray coating or by wet granulation techniques. In addition to naturally water-insoluble materials, some substrates of digestive enzymes can be treated with cross-linking procedures, resulting in the formation of non-soluble networks. Many methods of cross-linking proteins, initiated by both chemical and physical means, have been reported. One of the most common methods to obtain cross-linking is the use of chemical cross-linking agents. Examples of chemical cross-linking agents include aldehydes (gluteraldehyde and formaldehyde), epoxy compounds, carbodiimides, and genipin. In addition to these cross-linking agents, oxidized and native sugars have been used to cross-link gelatin. Cross-linking can also be accomplished using enzymatic means; for example, transglutaminase has been approved as a GRAS substance for cross-linking seafood products. Finally, cross-linking can be initiated by physical means such as thermal treatment, UV irradiation and gamma irradiation.

To produce a coating layer of cross-linked protein surrounding drug containing microparticles or drug particles, a water-soluble protein can be spray coated onto the microparticles and subsequently cross-linked by the one of the methods described above.

Alternatively, drug-containing microparticles can be microencapsulated within protein by coacervation-phase separation (for example, by the addition of salts) and subsequently cross-linked. Some suitable proteins for this purpose include gelatin, albumin, casein, and gluten.

Polysaccharides can also be cross-linked to form a water-insoluble network. For many polysaccharides, this can be accomplished by reaction with calcium salts or multivalent cations, which cross-link the main polymer chains. Pectin, alginate, dextran, amylose and guar gum are subject to cross-linking in the presence of multivalent cations. Complexes between oppositely charged polysaccharides can also be formed; pectin and chitosan, for example, can be complexed via electrostatic interactions.

### Enteral Formulations

Suitable oral dosage forms include tablets, capsules, solutions, suspensions, syrups, and lozenges. Tablets can be made using compression or molding techniques well known in the art. Gelatin or non-gelatin capsules can be prepared as hard or soft capsule shells, which can encapsulate liquid, solid, and semi-solid fill materials, using techniques well known in the art. Oral mRNA delivery using capsules is described in Abramson, et al., Matter, 5 (3): 975-987 (2022) (incorporated herein by reference), using branched hybrid poly(β-amino ester) mRNA nanoparticles.

Formulations may be prepared using a pharmaceutically acceptable carrier. As generally used herein “carrier” includes, but is not limited to, diluents, preservatives, binders, lubricants, disintegrators, swelling agents, fillers, stabilizers, and combinations thereof. Carrier also includes all components of the coating composition, which may include plasticizers, pigments, colorants, stabilizing agents, and glidants. Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

“Diluents”, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

“Binders” are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

“Lubricants” are used to facilitate tablet manufacture. Examples of suitable lubricants include, but are not limited to, magnesium stearate, calcium stearate, stearic acid, glycerol behenate, polyethylene glycol, talc, and mineral oil.

“Disintegrants” are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross-linked polymers, such as cross-linked PVP (Polyplasdone® XL from GAF Chemical Corp).

“Stabilizers” are used to inhibit or retard drug decomposition reactions, which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA).

### Controlled Release Enteral Formulations

Oral dosage forms, such as capsules, tablets, solutions, and suspensions, can for formulated for controlled release. For example, the one or more compounds and optional one or more additional active agents can be formulated into nanoparticles, microparticles, and combinations thereof, and encapsulated in a soft or hard gelatin or non-gelatin capsule or dispersed in a dispersing medium to form an oral suspension or syrup. The particles can be formed of the agent and a controlled release polymer or matrix. Alternatively, the agent particles can be coated with one or more controlled release coatings prior to incorporation into the finished dosage form.

In another embodiment, the one or more compounds and optional one or more additional active agents are dispersed in a matrix material, which gels or emulsifies upon contact with an aqueous medium, such as physiological fluids. In the case of gels, the matrix swells entrapping the active agents, which are released slowly over time by diffusion and/or degradation of the matrix material. Such matrices can be formulated as tablets or as fill materials for hard and soft capsules.

In still another embodiment, the one or more compounds, and optional one or more additional active agents are formulated into a sold oral dosage form, such as a tablet or capsule, and the solid dosage form is coated with one or more controlled release coatings, such as a delayed release coatings or extended-release coatings. The coating or coatings may also contain the compounds and/or additional active agents.

### Extended-Release Dosage Forms

The extended-release formulations are generally prepared as diffusion or osmotic systems, which are known in the art. A diffusion system typically consists of two types of devices, a reservoir, and a matrix, and is well known and described in the art. The matrix devices are generally prepared by compressing the agent with a slowly dissolving polymer carrier into a tablet form. The three major types of materials used in the preparation of matrix devices are insoluble plastics, hydrophilic polymers, and fatty compounds. Plastic matrices include, but are not limited to, methyl acrylate-methyl methacrylate, polyvinyl chloride, and polyethylene. Hydrophilic polymers include, but are not limited to, cellulosic polymers such as methyl and ethyl cellulose, hydroxyalkylcelluloses such as hydroxypropyl-cellulose, hydroxypropylmethylcellulose, sodium carboxymethylcellulose, and Carbopol® 934, polyethylene oxides and mixtures thereof. Fatty compounds include, but are not limited to, various waxes such as carnauba wax and glyceryl tristearate and wax-type substances including hydrogenated castor oil or hydrogenated vegetable oil, or mixtures thereof.

In certain preferred embodiments, the plastic material is a pharmaceutically acceptable acrylic polymer, including but not limited to, acrylic acid and methacrylic acid copolymers, methyl methacrylate, methyl methacrylate copolymers, ethoxyethyl methacrylates, cyanocthyl methacrylate, aminoalkyl methacrylate copolymer, poly(acrylic acid), poly(methacrylic acid), methacrylic acid alkylamine copolymer poly(methyl methacrylate), poly(methacrylic acid) (anhydride), polymethacrylate, polyacrylamide, poly(methacrylic acid anhydride), and glycidyl methacrylate copolymers.

In certain preferred embodiments, the acrylic polymer is comprised of one or more ammonio methacrylate copolymers. Ammonio methacrylate copolymers are well known in the art and are described in NF XVII as fully polymerized copolymers of acrylic and methacrylic acid esters with a low content of quaternary ammonium groups.

In one preferred embodiment, the acrylic polymer is an acrylic resin lacquer such as that which is commercially available from Rohm Pharma under the tradename EUDRAGIT t®. In further preferred embodiments, the acrylic polymer comprises a mixture of two acrylic resin lacquers commercially available from Rohm Pharma under the tradenames EUDRAGIT® RL30D and EUDRAGIT® RS30D, respectively. EUDRAGIT® RL30D and EUDRAGIT® RS30D are copolymers of acrylic and methacrylic esters with a low content of quaternary ammonium groups, the molar ratio of ammonium groups to the remaining neutral(meth)acrylic esters being 1:20 in EUDRAGIT® RL30D and 1:40 in EUDRAGIT® RS30D. The mean molecular weight is about 150,000. EUDRAGIT® S-100 and EUDRAGIT® L-100 are also preferred. The code designations RL (high permeability) and RS (low permeability) refer to the permeability properties of these agents. EUDRAGIT® RL/RS mixtures are insoluble in water and in digestive fluids. However, multi-particulate systems formed to include the same are swellable and permeable in aqueous solutions and digestive fluids.

The polymers described above such as EUDRAGIT® RL/RS may be mixed in any desired ratio in order to ultimately obtain a sustained-release formulation having a desirable dissolution profile. Desirable sustained release multi-particulate systems may be obtained, for instance, from 100% EUDRAGIT® RL, 50% EUDRAGIT® RL and 50% EUDRAGIT t® RS, and 10% EUDRAGIT® RL and 90% EUDRAGIT® RS. One skilled in the art will recognize that other acrylic polymers may also be used, such as, for example, EUDRAGIT® L.

Alternatively, extended-release formulations can be prepared using osmotic systems or by applying a semi-permeable coating to the dosage form. In the latter case, the desired agent release profile can be achieved by combining low permeable and high permeable coating materials in suitable proportion.

The devices with different agent release mechanisms described above can be combined in a final dosage form comprising single or multiple units. Examples of multiple units include, but are not limited to, multilayer tablets and capsules containing tablets, beads, or granules. An immediate release portion can be added to the extended release system by means of either applying an immediate release layer on top of the extended release core using a coating or compression process or in a multiple unit system such as a capsule containing extended and immediate release beads.

Extended-release tablets containing hydrophilic polymers are prepared by techniques commonly known in the art such as direct compression, wet granulation, or dry granulation. Their formulations usually incorporate polymers, diluents, binders, and lubricants as well as the active pharmaceutical ingredient. The usual diluents include inert powdered substances such as starches, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin, and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid, and hydrogenated vegetable oils.

Extended-release tablets containing wax materials are generally prepared using methods known in the art such as a direct blend method, a congealing method, and an aqueous dispersion method. In the congealing method, the agent is mixed with a wax material and either spray-congealed or congealed and screened and processed.

### Delayed Release Dosage Forms

Delayed release formulations can be created by coating a solid dosage form with a polymer film, which is insoluble in the acidic environment of the stomach, and soluble in the neutral environment of the small intestine. The delayed release dosage units can be prepared, for example, by coating an agent or an agent-containing composition with a selected coating material. The agent-containing composition may be, e.g., a tablet for incorporation into a capsule, a tablet for use as an inner core in a “coated core” dosage form, or a plurality of agent-containing beads, particles, or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit® (Rohm Pharma; Westerstadt, Germany), including EUDRAGIT® L30D-55 and L100-55 (soluble at pH 5.5 and above), EUDRAGIT® L-100 (soluble at pH 6.0 and above), EUDRAGIT® S (soluble at pH 7.0 and above, as a result of a higher degree of esterification), and EUDRAGITS® NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacctin, dimethyl phthalate, diethyl phthalate, dibutyl phthalate, dibutyl sebacate, triethyl citrate, tributyl citrate, triethyl acetyl citrate, castor oil and acctylated monoglycerides. A stabilizing agent is preferably used to stabilize particles in the dispersion.

Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

### Methods of Use

In various aspects, the present disclosure provides methods for treating a disease or disorder in a subject in need thereof. The methods of treatment are also provided and can be used alone or in combination with other methods disclosed herein such as the disclosed methods of detection, diagnosis, prognosis, and treatment monitoring. The methods typically include administering or providing to a subject in need thereof an effective amount of the disclosed anti-CLL-1 compositions and modalities, to treat the subject. In further forms, the disclosed methods and compositions can be delivered alone or in combination with a second immunotherapeutic, chemotherapeutic or active agent. In still further aspects, the subject in need thereof is a subject diagnosed with an autoimmune disease and the second therapeutic agent is a chemotherapeutic, preferably a cytotoxic agent. Different modalities are contemplated which provide immunotherapy with reduced side effects.

In further aspects, the subject has the disease or disorder. In some embodiments, the subject is suspected of having the disease or disorder. In some embodiments, the disease or disorder is an autoimmune disorder. In some embodiments, the disease or disorder is an inflammatory disorder. In some embodiments, the disease or disorder of the disclosed methods can be a CLL-1 (or CLEC12A)-expressing myeloid cell-mediated autoimmune disease or disorder. In some embodiments, the disease or disorder is a neurologic/neurodegenerative disease. In other embodiments, the disease or disorder is an EBY-associated lymphoproliferative disease.

In further aspects, the effective amount or therapeutically effective amount of the anti-CLL-1 compositions can be a dosage sufficient to inhibit pathologic inflammation, or reduce or deplete CLL-1-expressing pathologic immune cells.

In still further aspects, the composition can be depleted of CLL-1-expressing monocyte-derived macrophages and border-associated macrophages. In some forms, administration of the pharmaceutical composition results in neuroinflammation, reduced immune cell infiltration, and demyelination. For example, in some embodiments, the therapeutic effect of CLL-1-directed CAR-T cells in multiple sclerosis is mediated by depletion of CLL-1-expressing myeloid cells that sustain compartmentalized neuroinflammation. Progressive forms of multiple sclerosis can be characterized by inflammatory activity localized within the central nervous system behind an intact or partially intact blood-brain barrier, including perivascular cuffs, meningeal infiltrates, and subpial demyelination. Conventional immunosuppressive therapies targeting peripheral lymphocytes have limited efficacy against this compartmentalized process. In contrast, the CLL-1-expressing monocyte-derived macrophages, including border-associated macrophages, can infiltrate the central nervous system and replace yolk-sac-derived macrophages during disease, and that selective depletion of these CLL-1-positive cells by CAR-T therapy can reduce the drivers of neuroinflammation and demyelination. Accordingly, in some embodiments, treatment with CLL-1-directed CAR-T cells can reduce or prevent progression of multiple sclerosis by mitigating compartmentalized inflammatory activity within the central nervous system.

In some aspects, when administering the pharmaceutical composition, the dosage can be determined based on the amount effective to achieve an anti-immune effect in the recipient. For example, the composition can be administered in a dosage effective to reduce pathologic macrophage expansion, inhibit epitope spreading of autoreactive T and B cells, and/or prevent lesion progression. In other aspects, the pharmaceutical composition can decrease the number of CLL-1-positive macrophages cells, increase remyelination, and/or prevent axonal transection. In still other aspects, the compositions can be effective to increase the efficacy of disease-modifying therapy (DMT) agents such as anti-CD20 antibodies, SIP receptor modulators, or BTK inhibitors.

In yet other aspects, the compositions can be effective to increase the efficacy of disease-modifying therapies (DMTs) approved for multiple sclerosis, including, but not limited to, anti-CD20 antibodies (ocrelizumab, ofatumumab, ublituximab, rituximab), SIP receptor modulators (fingolimod, siponimod, ozanimod, ponesimod), fumarates (dimethyl fumarate, diroximel fumarate, monomethyl fumarate), interferons (interferon beta-la, interferon beta-1b, peginterferon beta-la), glatiramer teriflunomide, natalizumab, cladribine, alemtuzumab, BTK inhibitors, TYK2 inhibitors, etc.

The effective amount of the pharmaceutical composition can vary depending on multiple factors, including the species, age, weight, general health of the subject, severity of disease, and mode of administration. Thus, exact dosages may vary and should be determined by one of ordinary skill in the art using routine experimentation. For example, appropriate dosage regimens and administration schedules can be determined empirically based on in vitro and in vivo data. In some aspects, dosage ranges can be chosen to achieve inhibition and/or reduction of border-associated macrophage activation, prevent chronic lesion rim formation, and/or reduce CSF calprotectin levels.

Preferably, the dosage minimizes adverse side effects, such as off-target toxicity, unintended immune responses, or systemic genotoxicity. Dosage may vary depending on patient-specific factors, including age, overall health, route of administration, concurrent drug therapies, and disease type and stage. The dosage can be adjusted by the treating physician based on patient response. It is also recognized that the effective dosage may change over the course of treatment, requiring adjustments based on biomarker levels, such as calprotectin, MS progression, or treatment efficacy.

Dosage can be administered in single or multiple doses daily over the course of treatment. Dosing schedules can be determined by monitoring drug accumulation, pharmacokinetics, and biomarker responses in the patient. Persons skilled in the art can determine effective dosages, dosing methodologies, and repetition rates based on preclinical and clinical trial data. Dosages can vary based on the relative potency of specific nucleic acid compositions, which may be estimated using EC50 values from in vitro and in vivo disease models.

In some embodiments, the compositions are packaged in hermetically sealed containers to maintain stability and potency. In some forms, the compositions are supplied as a lyophilized powder or a water-free concentrate that can be reconstituted with a suitable diluent such as water or saline. Preferably, the composition is stored in a sterile, temperature-controlled environment to maintain biological activity. In alternative embodiments, the composition is supplied in liquid form at specified concentrations, ensuring consistent dosing for intravenous, intramuscular, subcutaneous, or oral administration. Formulations can also include stabilizers, excipients, or controlled-release carriers to increase bioavailability and therapeutic efficacy.

In some embodiments, the compositions for immunotherapy can be supplied in liquid form in a hermetically sealed container indicating the quantity and concentration of the active agent. Preferably, the liquid form of the compositions is supplied in a hermetically sealed container at a concentration of at least 1 mg/ml, more preferably at least 2.5 mg/ml, at least 5 mg/ml, at least 8 mg/ml, at least 10 mg/ml, at least 15 mg/ml, at least 25 mg/ml, at least 50 mg/ml, at least 100 mg/ml, at least 150 mg/ml, or at least 200 mg/ml. The composition can be formulated with stabilizers, excipients, or controlled-release agents to ensure effective bioavailability and therapeutic efficacy when administered via intravenous, intrathecal, subcutaneous, intramuscular, or oral routes.

In various aspects, the compositions can be effective to selectively deplete pathogenic subsets of immune cells. By depleting CLL-1-expressing monocyte-derived macrophages and border-associated macrophages, the composition can reduce chronic neuroinflammation, decreases lesion expansion, and/or prevent progression of disability. In still further aspects, the compositions can be used alone or in combination with existing disease-modifying therapy (DMT) regimens to improve treatment outcomes in patients with MS.

In some forms, the dosage of the compositions administered to a patient ranges from about 0.01 mg/kg to 100 mg/kg of the patient's body weight. Preferably, the dosage administered to a patient is between 0.01 mg/kg and 20 mg/kg, 0.01 mg/kg and 10 mg/kg, 0.01 mg/kg and 5 mg/kg, 0.01 mg/kg and 2 mg/kg, 0.01 mg/kg and 1 mg/kg, 0.01 mg/kg and 0.75 mg/kg, 0.01 mg/kg and 0.5 mg/kg, 0.01 mg/kg and 0.25 mg/kg, 0.01 mg/kg and 0.15 mg/kg, 0.01 mg/kg and 0.10 mg/kg, 0.01 mg/kg and 0.05 mg/kg, or 0.01 mg/kg and 0.025 mg/kg of the patient's body weight. In particular, the invention contemplates that the dosage administered to a patient is 0.2 mg/kg, 0.3 mg/kg, 1 mg/kg, 3 mg/kg, 6 mg/kg, or 10 mg/kg. A dose as low as 0.01 mg/kg may show appreciable pharmacodynamic effects. Dose levels of 0.10-1 mg/kg are predicted to be most appropriate, but higher doses (e.g., 1 mg/kg to 60 mg/kg) are also contemplated depending on the disease type, severity, and treatment regimen.

The pharmaceutical compositions can be administered as a single dose or in multiple doses at regular intervals. Injections and infusions of the disclosed compositions can be repeated as necessary until the desired therapeutic response is achieved. The effective dosage and treatment regimen for a particular patient can be determined by a clinician skilled in immunotherapy by monitoring the patient for inhibition of disease progression and overall treatment response. The frequency of administration may vary based on the severity and stage of disease, patient response, and combination with other therapies.

In some aspects, the unit dosage is in a form suitable for intravenous injection. In some aspects, the unit dosage is in a form for oral administration, inhalation, or intra-tumoral injection.

In some cases, the compositions is encapsulated in nanoparticles or other controlled-release delivery systems to facilitate bioavailability and pathologic immune cell targeting. The treatment can be continued for a time sufficient to achieve one or more therapeutic goals, such as reducing the number of pathologic immune cells, or stabilizing disease progression as measured by MRI lesion load and clinical disability scores.

In some aspects, administration is carried out daily, weekly, or at intervals over a specific period. Treatment regimens may be conducted over the course of two, three, four, or five days, weeks, or months, or may extend up to six months or more, including one year, two years, three years, or up to five years. The efficacy of a particular dose of the pharmaceutical composition can be assessed using objective clinical, histological, and imaging-based measures.

The success of treatment can be determined based on stabilization or slowing of disease progression, improvement in patient symptoms, and a reduced need for other medications or interventions. In some aspects, efficacy is measured as a reduction in Calprotectin levels at specific time points (e.g., 1-5 days, weeks, or months) following treatment. Additionally, combining the compositions with chemotherapy or other immunotherapy may further increase the therapeutic response and overall survival rates in patients.

### Modes of Administration

Any of the disclosed compositions can be used therapeutically in combination with a pharmaceutically acceptable carrier. The compositions described herein can be conveniently formulated into pharmaceutical compositions with a pharmaceutically acceptable carrier. These carriers can include sterile water, saline, buffered solutions at physiological pH, or lipid-based formulations to increase stability and bioavailability. Other therapeutic agents may be co-administered according to standard clinical procedures.

The pharmaceutical compositions can include, but are not limited to, carriers, thickeners, diluents, buffers, preservatives, surfactants, and stabilizers in addition to the active therapeutic agent. These compositions can be administered to the subject in a number of ways depending on whether local or systemic treatment is desired and the area to be treated. For example, a pharmaceutical composition can be administered as an intravenous infusion or directly injected into a tumor to maximize local bioavailability.

Additionally, the compositions can be administered orally, intravenously, intramuscularly, subcutaneously, intraperitoneally, intrathecal, intraarterially, intrathecally, or intralymphatically depending on the desired pharmacokinetics and treatment strategy. In some aspects, administration can be conducted via stereotactic delivery directly into affected tissue. The compositions can be formulated for sustained or controlled release to increase efficacy and reduce systemic toxicity.

Parenteral administration, if used, is generally characterized by injection or infusion. Injectables can be prepared in conventional forms, including liquid solutions, suspensions, emulsions, or solid forms suitable for reconstitution in liquid prior to injection. A sustained-release system may be used to maintain constant drug levels in circulation, reducing the frequency of administration.

Preparations for parenteral administration include sterile aqueous or non-aqueous solutions, suspensions, and emulsions that may contain buffers, diluents, and stabilizers.

Examples of non-aqueous solvents include propylene glycol, polyethylene glycol, vegetable oils (e.g., olive oil), and injectable organic esters (e.g., ethyl oleate). Aqueous carriers can include water, saline, emulsions, and buffered solutions. Parenteral vehicles can include sodium chloride solution, Ringer's lactate, dextrose solutions, and nutrient replenishers.

Additionally, preservatives and other excipients such as antimicrobials, antioxidants, chelating agents, and inert gases may be included to increase stability and prevent degradation.

The compositions can be co-administered with chemotherapeutic agents, or other immunotherapy, or targeted molecular inhibitors. In some embodiments, these compositions are encapsulated in liposomes, nanoparticles, or polymer-based carriers to increase cell targeting, uptake, and controlled release.

Administration of the compositions can be localized (e.g., direct injection, regional perfusion) or systemic (e.g., intravenous infusion, oral administration) depending on treatment goals and location. The dosage and frequency of administration can be adjusted based on symptom response, biomarker levels, and patient-specific factors. The efficacy of therapy including the compositions can be evaluated using symptom reduction, biomarker expression, histological analysis, and overall survival outcomes.

### Combination Therapies

Autoimmune diseases and disorders can be treated using a variety of localized and systemic therapeutic strategies, including targeted therapies, and immunotherapies. The selection of treatment depends on disease type, stage, genetic profile, the patient's overall health, and available medical resources.

The disclosed compositions can be administered alone or in combination with other standard immunotherapy treatments for autoimmune and neuroinflammatory diseases, including multiple sclerosis. The additional therapy may be administered simultaneously or sequentially as part of a combination treatment regimen. In other forms, the compositions can be effective to increase the efficacy of disease-modifying therapies (DMTs) approved for multiple sclerosis, including, but not limited to, anti-CD20 antibodies (ocrelizumab, ofatumumab, ublituximab, rituximab), SIP receptor modulators (fingolimod, siponimod, ozanimod, ponesimod), fumarates (dimethyl fumarate, diroximel fumarate, monomethyl fumarate), interferons (interferon beta-la, interferon beta-1b, peginterferon beta-la), glatiramer acetate, teriflunomide, natalizumab, cladribine, alemtuzumab, and BTK inhibitors.

The disclosed compositions can be administered in conjunction with standard immunotherapy treatments, including but not limited to alkylating chemotherapy (platinum-based agents, topoisomerase inhibitors, alkylating agents, antimetabolites, taxanes), targeted therapy (kinase inhibitors, PARP inhibitors, monoclonal antibodies, small-molecule inhibitors), and immunotherapy (immune checkpoint inhibitors, T-cell therapies, cytokine therapies). Other potential combinations include anti-angiogenesis therapy using VEGF inhibitors, COX-2 inhibitors, and tyrosine kinase inhibitors to reduce tumor vascularization and increase DNA damage accumulation in tumor cells.

The disclosed compositions can be co-administered with neuroprotective agents that promote oligodendrocyte survival and remyelination. The nucleic acid compositions can be applied across multiple immunotherapy types, including but not limited to CAR-T cells, CAR-NK cells, TCR-engineered T cells, bispecific antibodies, and RNA-based immunotherapies.

In some embodiments, the disclosed compositions comprise immune effector cells engineered to express a chimeric antigen receptor (CAR) that specifically binds CLL-1 (CLEC12A). The immune effector cells may include, without limitation, T lymphocytes (including CD4+, CD8+, regulatory, γδ, or iNKT subsets), natural killer (NK) cells, macrophages, dendritic cells, B cells, or stem cell-derived immune effector cells. In some embodiments, the CAR is introduced into macrophages (CAR-M), NK cells (CAR-NK), or induced pluripotent stem cell (iPSC)-derived immune cells, thereby enabling depletion of CLL-1-positive macrophages in the CNS by multiple effector cell lineages. Accordingly, the invention is not limited to CAR-T cells, but encompasses any CAR-engineered immune effector cell capable of targeting CLL-1+ myeloid populations associated with multiple sclerosis or related diseases.

### Subjects to be Treated

A subject in need of treatment is a subject diagnosed with or at risk of developing an autoimmune disorder or disease, including but not limited to multiple sclerosis and diseases of the like. Exemplary autoimmune disorder or disease that can be treated with the disclosed compositions include relapsing-remitting MS, secondary progressive MS, and primary progressive MS. MS is a heterogeneous disorder and can be classified into various clinical subtypes and phenotypes. Traditional classifications include relapsing-remitting MS (RRMS), secondary progressive MS (SPMS), primary progressive MS (PPMS), and progressive-relapsing MS (PRMS). More recent consensus criteria (e.g., 2013 revisions) recognize additional disease modifiers that distinguish phenotypes by activity and progression status. Thus, RRMS may be clinically isolated syndrome (CIS), active RRMS, or not active RRMS. Progressive MS may include PPMS or SPMS, each of which can be further characterized as (i) active with progression, (ii) active without progression, (iii) not active with progression, or (iv) not active without progression (stable disease). Accordingly, the methods described herein encompass treatment of all MS subtypes and phenotypes, including but not limited to CIS, RRMS, SPMS (active or inactive; with or without progression), PPMS (active or inactive; with or without progression), and PRMS. These embodiments ensure that therapeutic use of CLL-1-directed CAR-T cells is not limited by disease stage, activity, or classification scheme, and are intended to include any clinical variant or diagnostic formulation of MS recognized presently or in the future.

In some aspects, the subject is a mammal, including but not limited to humans, domesticated animals, farm animals, prize animals, and pets. Examples of farm animals include cattle, pigs, sheep, and goats. Prize animals can include show horses and thoroughbred racing horses. Companion animals such as dogs, rabbits, and cats can also develop autoimmune diseases similar to those found in humans and may benefit from the disclosed compositions. Preferably, the subject is human.

All methods and uses described herein can also include the step of identifying and selecting a subject in need of treatment or a subject who would benefit from administration of the disclosed compositions. The selection process may involve molecular diagnostics, biomarker analysis, genetic testing, or assessment of CSF calprotectin levels, neurofilament light chain, and paramagnetic rim lesion activity on MRI. In some aspects, the disclosed compositions may be used as a first-line treatment, adjunct therapy, or salvage therapy for relapsed or refractory diseases.

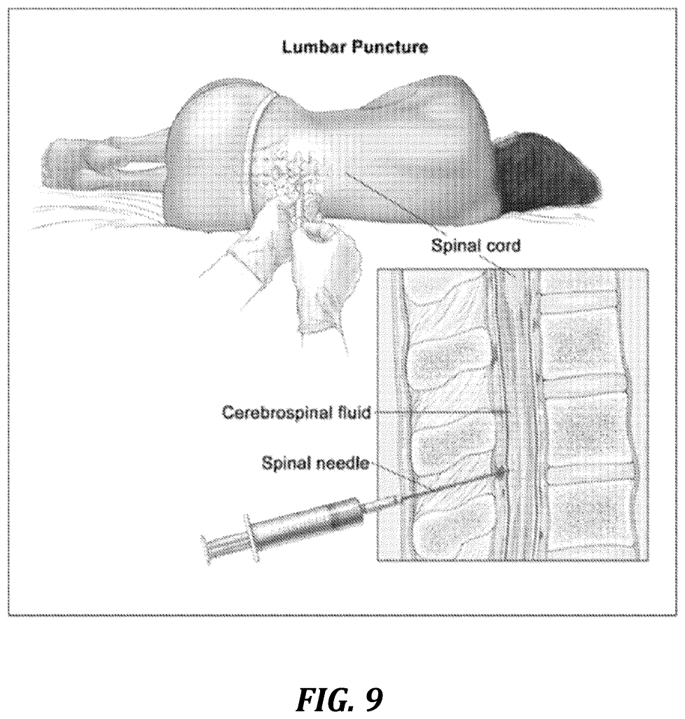

In certain embodiments, subjects may be selected for treatment based on disease severity, progression status, or functional disability as determined by one or more standardized clinical rating scales used in multiple sclerosis. For example, FIG. 9 depicts a lumbar puncture procedure for obtaining cerebrospinal fluid (CSF) samples from a subject. Such procedures enable measurement of biomarkers, including calprotectin, facilitating patient selection and monitoring of therapeutic response in accordance with the methods and systems disclosed herein.

### Controls

In certain embodiments, clinical evaluation of the disclosed CAR-T cell therapies in multiple sclerosis is performed using placebo-controlled designs adapted for cell therapy. Unlike small-molecule or oral DMT comparators, where placebo can be administered as an inert pill, CAR-T therapy requires an infusion procedure. Accordingly, placebo control may comprise a sham or inactive infusion designed to mimic the procedural aspects of CAR-T administration, thereby maintaining blinding of subjects and investigators. In some embodiments, a double-dummy design is employed in which all trial participants receive both an infusion (active CAR-T or sham placebo) and a comparator agent (active DMT or dummy control), ensuring that neither the subject nor the investigator can distinguish treatment allocation. Such designs preserve methodological rigor and permit unbiased assessment of efficacy and safety in the context of advanced MS therapeutics. In certain embodiments, the efficacy of the disclosed compositions is evaluated in a randomized, double-blind, placebo-controlled clinical trial, optionally using a double-dummy design, wherein the placebo comprises a sham or inactive infusion procedure designed to mimic CAR-T cell administration.

The therapeutic efficacy of the disclosed compositions and pharmaceutical formulations can be evaluated by comparison to a control. Suitable controls are well known in the art. A typical control involves assessing a condition or symptom of a subject before and after administration of the described compositions and pharmaceutical formulations. The condition or symptom can be a biochemical, molecular, physiological, or pathological marker. For example, the effect of the described compositions on CSF calprotectin levels, disease progression, or patient survival can be compared to an untreated subject, or to the condition of the subject prior to treatment. In some aspects, neurofilament light chain (NfL), or other pharmacologic or physiologic indicators are measured in a subject prior to treatment, and again at one or more time points following initiation of treatment. In some aspects, the control is a reference level or an average determined by measuring population-based biomarker norms or survival rates in subjects without the disease or in subjects receiving standard-of-care treatment. In other aspects, the efficacy of the disclosed compositions is compared to a conventional treatment such as anti-CD20 antibodies (ocrelizumab, ofatumumab, ublituximab, rituximab), SIP receptor modulators (fingolimod, siponimod, ozanimod, ponesimod), fumarates (dimethyl fumarate, diroximel fumarate, monomethyl fumarate), interferons (interferon beta-la, interferon beta-1b, peginterferon beta-la), glatiramer acetate, teriflunomide, natalizumab, cladribine, alemtuzumab, and BTK inhibitors.

The actual effective amounts of the composition can vary according to factors including the specific compositions administered, the particular formulation used, the mode of administration, and the age, weight, and condition of the subject being treated. Additional factors such as the route of administration and the type, stage, and molecular characteristics of the disease also influence the effective dose.

In some aspects, the disclosed compositions increase survival rates or reduce disease incidence in a treated subject compared to an untreated control by more than 1%, such as by 5%, 10%, 20%, 30%, 40%, 50%, 60%, 75%, 100%, 150%, 200%, or up to 300%. In other aspects, the compositions reduce the incidence or severity of one or more symptoms of multiple sclerosis, such as fatigue, spasticity, or cognitive decline, as compared to an untreated control by up to 5%, 10%, 20%, 30%, 40%, 50%, 60%, 75%, or 100%. Preferably, the disclosed compositions specifically target CLL-1 expressing immune cells while having minimal or no effect on healthy cells, thereby reducing potential off-target toxicity and adverse effects. It is to be understood that the disclosed method and compositions are not limited to specific synthetic methods, specific analytical techniques, or to particular reagents unless otherwise specified, and, as such, can vary. It is also to be understood that the terminology used herein is for the purpose of describing particular embodiments only and is not intended to be limiting.

In further aspects, the present disclosure provides methods of treating multiple sclerosis by administering engineered CAR-T cells targeting CLL-1. These methods may be applicable to various forms of MS, including relapsing-remitting MS (RRMS), secondary progressive MS (SPMS), and primary progressive MS (PPMS).

In some embodiments, the method can comprise one or more of the following steps:
Patient Selection: Identifying MS patients who may benefit from anti-CLL-1 CAR-T cell therapy. This may include patients with evidence of smoldering neuroinflammation not adequately controlled by existing therapies. Lymphodepletion: Administering a lymphodepletion regimen prior to CAR-T cell infusion. This may comprise fludarabine (e.g., 25 mg/m²/day for 3 days) and cyclophosphamide (e.g., 200 mg/kg for 2 days) to create space for CAR-T cell expansion and persistence

In certain embodiments, the lymphodepletion regimen comprises one or more agents selected from fludarabine, cyclophosphamide, bendamustine, cladribine, alemtuzumab, or mitoxantrone (Novantrone®), or combinations thereof, administered prior to CAR-T infusion.

CAR-T Cell Infusion: Administering a single dose of anti-CLL-1 CAR-T cells intravenously. The CAR-T cells may circulate through the bloodstream, cross the BCSFB, and target CLL-1-expressing BAMs in the choroid plexus and other CNS border regions. Monitoring: Regular assessment of safety, tolerability, and efficacy at predefined timepoints (e.g., day 28, 3 months, 6 months, 9 months, 12 months). This may include neurological examinations, MRI scans, measurement of CAR-T cell persistence, and assessment of calprotectin levels in cerebrospinal fluid.

The anti-CLL-1 CAR-T cells may target and eliminate CLL-1-expressing BAMs through various mechanisms: Recognition and Binding: The anti-CLL-1 CAR binds to CLL-1 expressed on BAMs, forming an immunological synapse between the CAR-T cell and the target cell. Activation: Upon CLL-1 binding, the CAR transmits activation signals through its intracellular domain, leading to CAR-T cell activation and effector functions. Elimination: The activated CAR-T cell releases cytotoxic molecules such as perforin and granzymes that induce apoptosis in the CLL-1-expressing target cell. By depleting CLL-1-expressing BAMs, anti-CLL-1 CAR-T cells may disrupt the smoldering neuroinflammation pathway, potentially reducing pro-inflammatory cytokine production, preventing further recruitment of peripheral immune cells, and halting disease progression.

In further aspects, the present disclosure provides various embodiments wherein calprotectin is used as a Biomarker, for example, for Monitoring Response to Anti-CLL-1 CAR-T Cell Therapy. In still further aspects, calprotectin, a heterodimeric protein complex consisting of S100A8 and S100A9, is primarily released from neutrophils, monocytes, and macrophages during inflammation. Elevated levels of calprotectin in cerebrospinal fluid (CSF) can indicate active neuroinflammation in MS, particularly smoldering neuroinflammation mediated by BAMs.

Thus, according to various aspects of the present disclosure, provided herein are methods of diagnosis, disease progression, and/or monitoring treatment response in CLL-1 (or CLEC12A)-expressing myeloid cell-mediated autoimmune diseases and disorders by measuring calprotectin levels. In further aspects, the present disclosure provides methods of monitoring response to anti-CLL-1 CAR-T cell therapy by measuring calprotectin levels in CSF before and after treatment. A decrease in calprotectin levels can indicate a reduction in BAM-mediated inflammation and a positive response to therapy.

In some embodiments, the method can comprise one or more of the following: Baseline Measurement: Collecting CSF via lumbar puncture (FIG. 9) before anti-CLL-1 CAR-T cell therapy and measuring calprotectin levels. Treatment: Administering anti-CLL-1 CAR-T cell therapy as described above. Follow-up Measurements: Collecting CSF at predefined timepoints after treatment and measuring calprotectin levels. Assessment: Comparing post-treatment calprotectin levels to baseline levels to assess treatment response. A significant decrease in calprotectin levels may indicate effective depletion of BAMs and reduction in neuroinflammation.

In still further aspects, calprotectin monitoring can provide an objective, quantitative measure of treatment efficacy that complements clinical and radiological assessments. It can help identify treatment responders and non-responders, guide treatment decisions, and provide insights into the underlying pathophysiological processes.

In some embodiments, calprotectin is used to: Monitor Disease Relapses: Elevated calprotectin levels can be detected in RRMS patients during relapses, indicating increased neuroinflammation. Regular monitoring of calprotectin can help identify relapse onset and adjust therapy accordingly. Track Disease Progression: In SPMS and PPMS, where relapses are less frequent or absent, calprotectin levels can still reflect ongoing immune activity and chronic neuroinflammation. Monitoring these levels helps track disease progression and assess the efficacy of therapeutic interventions. Assess Therapeutic Response: In some embodiments, calprotectin can be used to evaluate the effectiveness of therapeutic interventions, such as CAR-T cell therapy, monoclonal antibodies, bi-specific antibodies, or ADCs targeting CLEC12A. A reduction in calprotectin levels after treatment indicates a successful reduction in immune activation and neuroinflammation.

CLL-1 (or CLEC12A)-Targeting CAR-T Cell Therapy: As detailed previously, CAR-T cell therapy targeting CLL-1 (or CLEC12A) can be administered to patients across various stages of MS to selectively deplete myeloid cells involved in disease progression. In some embodiments, calprotectin levels can be monitored alongside CAR-T cell therapy to gauge treatment efficacy.

In some embodiments, the subject has or is suspected of having an autoimmune disorder, an inflammatory disorder, neurologic/neurodegenerative diseases, and/or EBV-associated Lymphoproliferative Disease. In some embodiments, the subject has or is suspected of having an autoimmune disease. Non-limiting examples of an autoimmune disease or disorder include Achalasia, Addison's disease, Adult Still's disease, Agammaglobulinemia, Alopecia arcata, Amyloidosis, Ankylosing spondylitis, Anti-GBM/Anti-TBM nephritis, Anti-MOG Antibody-associated disease, Antiphospholipid syndrome, Autism, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Bal6 disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangitis, Graves' disease, Guillain-Barre syndrome (including Miller Fisher Syndrome), Hashimoto's thyroiditis, Hemolytic anemia, Henoch-Schonlein purpura (HSP), Herpes gestationis or pcmphigoid gestationis (PG), Hidradenitis Suppurativa (HS) (Acne Inversa), Hypogammalglobulinemia, IgA Nephropathy, IgG4-related sclerosing disease, Immune thrombocytopenia purpura (ITP), Inclusion body myositis (IBM), Interstitial cystitis (IC), Juvenile arthritis, Juvenile diabetes (Type I diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple Sclerosis (including Relapsing Forms of MS and/or Progressive Forms of MS, and/or Radiographically Isolated Syndrome and/or Clinically Isolated Syndrome), Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Parancoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, II, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenia purpura (TTP), Tolosa-Hunt syndrome (THS), Transverse myelitis, Type I diabetes, Ulcerative colitis (UC), Undifferentiated connective tissue disease (UCTD), Uveitis, Vasculitis, Vitiligo, and Vogt-Koyanagi-Harada Disease.

In some embodiments, the subject has or is suspected of having an inflammatory disease. Non-limiting examples of inflammatory diseases include allergy, ankylosing spondylitis, asthma, atopic dermatitis, autoimmune diseases or disorders, celiac disease, chronic obstructive pulmonary disease (COPD) (including chronic bronchitis and emphysema), chronic peptic ulcer, cystic fibrosis, diabetes (e.g., type I diabetes and type 2 diabetes), Familial Mediterranean Fever (FMF), glomerulonephritis, gout, hepatitis (e.g., active hepatitis), inflammatory bowel disease (IBD) such as Crohn's disease or ulcerative colitis, myositis, osteoarthritis, pelvic inflammatory disease (PID), multiple sclerosis, neurodegenerative diseases of aging, periodontal disease (e.g., periodontitis), pre-perfusion injury transplant rejection, progressive rubella panencephalitis (PRP), psoriasis, rheumatic disease, scleroderma, sinusitis, subacute sclerosing panencephalitis (SSPE), and tuberculosis.

In some embodiments, the subject has or is suspected of having a neurological disorder/neurodegenerative disorder. Non-limiting examples of neurological diseases include ADD (Attention-Deficit Disorder), ADHD (Attention-Deficit Hyperactivity Disorder), Asperger Syndrome, Autism Spectrum Disorder, Amyotrophic Lateral Sclerosis, Alzheimer's Syndrome, Brown-Sequard Syndrome, Cerebral Palsy, Guillain-Barre Syndrome, Gaucher Disease, Huntington's Disease, Multiple Sclerosis (MS), Muscular Dystrophy, and Spinal Muscular Atrophy.

In some embodiments, the subject has or is suspected of having a white matter inflammatory or demyelinating disease. Non-limiting examples of such diseases include multiple sclerosis (MS), neuromyelitis optica spectrum disorder (NMOSD), myelin oligodendrocyte glycoprotein antibody-associated disease (MOGAD), acute disseminated encephalomyelitis (ADEM), optic neuritis, transverse myelitis, Balo's concentric sclerosis, Schilder's disease, Marburg's variant MS, tumefactive demyelinating disease, Susac's syndrome, anti-aquaporin-4 antibody-associated disorders, anti-MOG antibody-associated disorders, and other antibody-mediated white matter diseases. Additional conditions include post-infectious or post-vaccination demyelination, progressive multifocal leukoencephalopathy (PML), leukodystrophies with inflammatory features, and other disorders characterized by immune-mediated injury to CNS white matter. These examples are provided for illustration and are not intended to limit the scope of the invention.

In some embodiments, the subject has or is suspected of having an EBV-Associated Lymphoproliferative Disease (e.g., EBV+LPD). Non-limiting examples of EBV+LPDs include EBY-associated reactive lymphoid proliferations, Epstein-Barr virus-positive reactive lymphoid hyperplasia, Epstein-Barr virus-positive infectious mononucleosis, Epstein-Barr virus-related hemophagocytic lymphohistiocytosis, Chronic active Epstein-Barr virus infection, Severe mosquito bite allergy, Hydroa vacciniforme-like lymphoproliferative disease, Epstein-Barr virus-positive mucocutaneous ulcer, Non-Hodgkin's Lymphomas (NHL), EBV+B cell lymphoproliferative diseases, Epstein-Barr virus-positive Burkitt lymphoma, Epstein-Barr virus-positive lymphomatoid granulomatosis, Epstein-Barr virus-positive Hodgkin lymphoma, Epstein-Barr virus-positive diffuse large B cell lymphoma, not otherwise specified, Epstein-Barr virus-associated diffuse large B cell lymphoma associated with chronic inflammation, Fibrin-associated diffuse large B cell lymphoma, Epstein-Barr virus-positive human herpes virus 8-associated B cell lymphoproliferative disorders, Primary effusion 1 virus-positive, human herpes virus-positive germinotropic lymphoproliferative disorder, Epstein-Barr virus-positive plasmablastic lymphoma, Epstein-Barr virus-associated plasma cell myeloma, EBV+ NK/T cell lymphoproliferative diseases, Peripheral T-cell lymphomas, Extranodal NK/T cell lymphoma, nasal type, Epstein-Barr virus-associated peripheral T cell lymphoma, Angioimmunoblastic T cell lymphoma, Follicular T cell lymphoma, Systemic Epstein-Barr virus-positive T cell lymphoma of childhood, Epstein-Barr virus-associated aggressive NK cell leukemia, Intravascular NK/T-cell lymphomas, EBY-associated immunodeficiency-related lymphoproliferative disorders, EBY-related and HIV-related LPD, Post-transplant lymphoproliferative disorders), EB V-associated histiocytic-dendritic disorders, and Inflammatory pseudotumor-like follicular/fibroblastic dendritic cell sarcoma.

Although many embodiments described herein focus on CLL-1 (CLEC12A), the invention is not limited thereto. In some embodiments, the target antigen may be any C-type lectin family member expressed on monocyte-derived or disease-associated macrophages that infiltrate the CNS and replace yolk sac-derived BAMs. The human C-type lectin domain family includes, without limitation, CLEC1 through CLEC16, as well as related subgroups such as CLEC2 (CLEC2A, CLEC2B), CLEC4 family members (CLEC4A-CLEC4G), CLEC5A, CLEC6A, CLEC7A (Dectin-1), CLEC9A, CLEC10A, CLECIIA, CLEC12A (CLL-1), CLEC14A, CLEC16A, and other functionally similar lectins. Any such C-type lectin that is selectively upregulated on pathogenic macrophages, relative to yolk sac-derived BAMs, is within the scope of the present disclosure.

According to the Presented herein are aspects relevant to this disclosure:

In one aspect, a therapeutic product may comprise engineered CAR-T cells that express a CAR comprising an scFv specific for CLL-1, a hinge domain, a transmembrane domain, and an intracellular signaling domain selected from CD3ζ with a costimulatory domain such as 4-1BB, CD28, or OX40.

In another aspect, the engineered CAR-T cells may comprise one or more genome edits selected from TCR knockout, PD-1 knockout, B2M knockout, or B2M-HLA-E knock-in. In another aspect, the engineered CAR-T cells may further comprise edits reducing expression of checkpoint inhibitors selected from PD-1, CTLA-4, LAG-3, and TIM-3.

In another aspect, a pharmaceutical composition may comprise CLL-1 CAR-T cells formulated with a pharmaceutically acceptable carrier.

In another aspect, a kit may comprise one or more vials of CLL-1 CAR-T cells and instructions for use in treating multiple sclerosis or related autoimmune diseases.

In another aspect, a system may comprise: a delivery apparatus for CAR-T infusion; a sampling apparatus for obtaining cerebrospinal fluid or other biological fluids; an analysis unit for calprotectin measurement; and a controller configured to implement dosing and monitoring protocols, wherein the system may further comprise displays, databases of reference biomarker levels, or modules configured to integrate biomarker data with clinical decision support.

In another aspect, diagnosing or monitoring a subject with an autoimmune disease characterized by replacement of yolk sac-derived macrophages with monocyte-derived macrophages may comprise: obtaining a disease-relevant biological fluid from a subject, the biological fluid selected from extracellular fluid, transcellular fluid, cerebrospinal fluid (CSF), synovial fluid, saliva, stool, and urine; measuring a concentration of calprotectin in the biological fluid; and performing a clinical procedure based on the measured concentration of calprotectin, wherein elevated calprotectin indicates compartmentalized inflammation driven by CLL-1-expressing monocyte-derived macrophages.

In another aspect, the clinical procedure may comprise diagnosing disease presence, activity, or progression, adjusting a stage of a clinical trial, or administering a therapeutic agent.

In another aspect, a decrease in the concentration of calprotectin following therapy may indicate a positive treatment response. In another aspect, the concentration of calprotectin may be compared to a previously measured concentration from the same subject. In another aspect, the concentration of calprotectin may be compared to a threshold concentration determined from a population of patients. In another aspect, the autoimmune disease may be multiple sclerosis and the disease-relevant biological fluid may be cerebrospinal fluid. In another aspect, an elevated or sustained calprotectin concentration may indicate progressive multiple sclerosis. In another aspect, the concentration of calprotectin may be correlated with clinical outcomes including disability progression, relapse rate, or MRI lesion activity. In another aspect, the concentration of calprotectin may be combined with neuroimaging data or additional biomarkers to enhance prediction of progression.

In another aspect, enriching or stratifying subjects in a clinical trial may comprise: measuring calprotectin in a disease-relevant biological fluid; and preferentially enrolling subjects with calprotectin above a predetermined threshold, wherein the method captures progressive disease biology. In another aspect, calprotectin may be combined with CLL-1 expression on macrophages, MRI findings, or additional biomarkers for trial inclusion. In another aspect, enrichment may be performed by MS subtype selected from RRMS, SPMS, or PPMS. In another aspect, longitudinal calprotectin data may serve as a pharmacodynamic marker of therapy. In another aspect, obtaining regulatory qualification of calprotectin as a biomarker may comprise: assembling datasets correlating calprotectin with disease progression, therapy response, or clinical outcomes; establishing threshold values distinguishing stable from progressive disease or defining clinically meaningful treatment response; and submitting a biomarker qualification package including analytical validation of calprotectin measurement to a regulatory agency.

In another aspect, guiding clinical development of a therapeutic candidate for progressive multiple sclerosis may comprise: enrolling subjects with progressive multiple sclerosis who are free of clinical relapses during a prespecified lead-in period; collecting biological samples at baseline and at one or more on-treatment timepoints during a Phase 2 clinical trial of twelve to twenty-four weeks duration; quantifying calprotectin concentration in the samples, wherein calprotectin comprises the S100A8/S100A9 heterodimer; determining treatment effect by comparing change from baseline in treated subjects relative to a control cohort; and declaring a go/no-go decision for advancement to a Phase 3 trial based on a prespecified calprotectin criterion that is independent of relapse rate, gadolinium-enhancing lesion counts, and systemic inflammatory markers.

In another aspect, cerebrospinal fluid obtained by lumbar puncture, with calprotectin measured by immunoassay, including enzyme-linked immunosorbent assay, electrochemiluminescence, or immunoturbidimetry. In another aspect, cerebrospinal fluid, serum, plasma, or a paired cerebrospinal fluid and serum set, with treatment effect normalized to a cerebrospinal fluid/serum albumin quotient. In another aspect, the on-treatment timepoint of aspect 21 may occur at week 12, week 16, or week 24. In another aspect, prespecified calprotectin criterion may comprise at least a 15% reduction from baseline exceeding the mean change in the control cohort by at least 10%, or an absolute reduction of at least 150 ng/m. In another aspect, subjects with corticosteroid exposure, acute infection, or clinical relapse within eight weeks of sampling may be excluded from analysis. In another aspect, the control cohort may comprise placebo. In another aspect, the progressive multiple sclerosis may be non-relapsing secondary progressive multiple sclerosis or primary progressive multiple sclerosis. In another aspect, a Bayesian decision rule may be applied, declaring a go decision when the posterior probability that the therapeutic candidate reduces calprotectin versus control exceeds 0.80.

In another aspect, a go/no-go decision may trigger at least one Phase 3 design action, including sample-size determination, enrichment for subjects with elevated baseline calprotectin, or stratification by baseline calprotectin. In another aspect, calprotectin analysis may be performed in a central laboratory under predefined acceptance criteria for sample handling, freeze-thaw cycles, and assay calibration. In another aspect, a go/no-go decision may be rendered without reliance on relapse counts, gadolinium-enhancing lesion counts, neurofilament light chain, or glial fibrillary acidic protein.

In an aspect, a method of treating multiple sclerosis in a subject, comprising administering to the subject an engineered T cell that specifically binds to CLL-1 under conditions effective to deplete monocyte-derived macrophages that infiltrate the central nervous system via CCR2+ monocyte recruitment. In an aspect, the monocyte-derived macrophages localize to perivascular spaces. In an aspect, the monocyte-derived macrophages infiltrate a choroid plexus stroma. In an aspect, the monocyte-derived macrophages infiltrate the dural stroma. In an aspect, the monocyte-derived macrophages infiltrate the leptomeningeal space. In an aspect, the depletion of monocyte-derived macrophages prevents replacement of yolk sac-derived border-associated macrophages. In an aspect, the subject is diagnosed with progressive multiple sclerosis. In an aspect, the subject is selected for treatment based on disease severity or progression as determined by a standardized clinical disability scale.

In certain embodiments, the disclosed methods are applied to subjects with progressive multiple sclerosis, including secondary progressive MS (SPMS) and primary progressive MS (PPMS). Progressive forms of MS are characterized by compartmentalized neuroinflammation behind an intact or partially intact blood-brain barrier, sustained by CLL-1-expressing border-associated macrophages and other monocyte-derived myeloid cells. Administration of CLL-1-directed CAR-T cells provides a therapeutic strategy to deplete these pathogenic populations, thereby reducing demyelination and disease progression in progressive MS.

### EXAMPLES

The following prophetic examples are intended to illustrate certain aspects of the present disclosure, but do not exemplify the full scope of the disclosure.

### Example 1: Generation of Anti-CLL-1 CAR Constructs

Anti-CLL-1 CAR constructs can be generated using a single-chain variable fragment (scFv) derived from a monoclonal antibody that specifically binds CLL-1. The scFv can be linked to a CD8α hinge and transmembrane domain, followed by a 4-1BB costimulatory domain and a CD3ζ signaling domain. The anti-CLL-1 CAR construct can be cloned into a lentiviral vector under the control of a human EFla promoter. The lentiviral vector also can contain a T2A sequence followed by a truncated EGFR (EGFRt) to enable detection and potential enrichment of transduced cells.

Lentiviral particles can be produced by transfecting HEK293T cells with the anti-CLL-1 CAR lentiviral vector and packaging plasmids. Viral supernatants can be collected, concentrated, and titrated on target cells.

### Example 2: Engineering of CB-012 Cells

Primary human T cells can be isolated from peripheral blood mononuclear cells (PBMCs) obtained from healthy donors. T cells can activated using anti-CD3/CD28-coated beads and cultured in the presence of IL-2. Activated T cells can be transduced with the anti-CLL-1 CAR lentiviral vector.

Transduction efficiency can be assessed by flow cytometry detection of EGFRt expression.

CRISPR-Cas9 gene editing can be used to introduce additional genetic modifications:
TCR knockout: TRAC gene can be targeted to eliminate endogenous TCR expression
PD-1 knockout: PDCDI gene can be targeted to eliminate PD-1 expression
B2M knockout: B2M gene can be targeted to eliminate beta-2-microglobulin expression
B2M-HLA-E insertion: A B2M-HLA-E fusion construct can be inserted at the B2M locus

Edited T cells can be expanded in the presence of IL-2, IL-7, and IL-15. The expanded cells can be harvested, washed, and cryopreserved for later use. The final CB-012 product can be characterized for CAR expression, genetic modifications, T cell phenotype, and functional activity against CLL-1-expressing target cells.

### Example 3: Generation of Anti-CLL-1 CAR-T Cells (CB-012)

In various embodiments, anti-CLL-1 CAR-T cells (e.g., CB-012) can be generated using standard genetic engineering techniques. Briefly, T cells may be isolated from peripheral blood mononuclear cells (PBMCs) obtained from healthy donors. The isolated T cells can be activated using anti-CD3/CD28 stimulation and transduced with a viral vector encoding the anti-CLL-1 CAR.

For allogeneic CAR-T cells, additional genetic modifications can be performed using CRISPR-Cas9 gene editing to:
Knock out the TCR to prevent graft-versus-host disease
Knock out PD-1 to prevent T cell exhaustion
Knock out B2M to prevent MHC class I expression and reduce host immune recognition
Insert a B2M-HLA-E fusion to prevent NK cell-mediated killing

The genetically modified T cells may be expanded in culture and formulated for clinical use. Quality control testing may be performed to ensure product purity, potency, and safety.

### Example 4: In Vitro Functional Assessment Anti-CLL-1 CAR-T cells (CB-012) Activity Against CLL-1-Expressing Cells

CB-012 anti-CLL-1 CAR-T cells can be tested for specific recognition and killing of CLL-1-expressing target cells in vitro.

Target cells can include:
Cell lines engineered to express CLL-1
Primary human macrophages expressing CLL-1
Choroid plexus macrophages isolated from post-mortem human brain tissue

CB-012 cells can be co-cultured with target cells at various effector-to-target ratios. Cytotoxic activity can be assessed by measuring target cell viability, release of cytokines (IFNγ, TNFα, IL-2), and degranulation markers (CD107a).

Control conditions can include:
Untransduced T cells
T cells expressing an irrelevant CAR
CB-012 cells co-cultured with CLL-1-negative target cells

CB-012 cells are expected to demonstrate specific recognition and killing of CLL-1-expressing target cells, with minimal activity against CLL-1-negative cells. The cytotoxic activity is expected to be associated with production of pro-inflammatory cytokines and expression of degranulation markers.

### Example 5: Preclinical Evaluation of Anti-CLL-1 CAR-T Cells in MS Models

In further aspects, the efficacy of anti-CLL-1 CAR-T cells may be evaluated in preclinical models of MS, such as experimental autoimmune encephalomyelitis (EAE). EAE may be induced in mice by immunization with myelin oligodendrocyte glycoprotein (MOG) peptide, and anti-CLL-1 CAR-T cells may be administered intravenously at various timepoints.

Clinical scores, weight loss, and survival may be monitored to assess disease severity. Immunohistochemical analysis of CNS tissues may be performed to evaluate BAM depletion, inflammation, and demyelination. CSF may be collected to measure calprotectin levels and correlate with disease activity and treatment response.

### Example 6: Preclinical Assessment of CB-012 Efficacy in an MS Animal Model

In further aspects, efficacy of CB-012 anti-CLL-1 CAR-T cells can be assessed in a relevant animal model of MS.

Experimental autoimmune encephalomyelitis (EAE) can be induced in humanized mice expressing human CLL-1 on macrophages. Once disease is established, mice can be treated with:
CB-012 anti-CLL-1 CAR-T cells
Control CAR-T cells targeting an irrelevant antigen
Untransduced T cells
Vehicle control

Disease progression can be monitored by:
Clinical scoring of neurological symptoms
Histopathological assessment of CNS inflammation and demyelination
Flow cytometric analysis of immune cell populations in CNS and periphery
Measurement of inflammatory cytokines in CSF and serum

CB-012-treated mice is expected to show reduced disease severity compared to control groups. This was associated with specific depletion of CLL-1-expressing macrophages in the choroid plexus and other CNS border regions, reduced neuroinflammation, and preservation of myelin.

### Example 7: Clinical Trial Design for Anti-CLL-1 CAR-T Cell Therapy in MS

In further aspects, a phase 1 clinical trial can be designed to evaluate the safety, tolerability, and preliminary efficacy of anti-CLL-1 CAR-T cell therapy (e.g., CB-012) in patients with MS. The trial can include patients with RRMS who have failed at least two disease-modifying therapies, as well as patients with SPMS or PPMS with evidence of active inflammation.

Key eligibility criteria may include:
Adults≥18 years of age
Evidence of active inflammation on MRI or elevated cerebrospinal fluid markers
Evidence of smoldering neuroinflammation not adequately controlled by existing therapies
For allogeneic CAR-T cells: HLA matching of ≥4 alleles with absence of pre-infusion donor-specific antibodies (DSA)
Adequate organ function
Confirmed diagnosis of MS (RRMS, SPMS, or PPMS)

The trial design can include separate cohorts for different MS subtypes:
Cohort 1: Relapsing-remitting MS (RRMS)
Cohort 2: Secondary progressive MS (SPMS)
Cohort 3: Primary progressive MS (PPMS)

Treatment regimen can include:
lymphodepletion regimen comprising fludarabine (25 mg/m²/day for 3 days) and cyclophosphamide (200 mg/kg for 2 days) prior to CAR-T cell infusion.
A single dose of CB-012 may be administered intravenously on day 0
Assessments: patients may be monitored for safety, tolerability, and efficacy at day 28, 3 months, 6 months, 9 months, and 12 months.

Primary endpoints can include:
Safety and tolerability
Incidence and severity of adverse events, including cytokine release syndrome and neurotoxicity

The present invention includes at least the following aspects:

Aspect 1: A method of treating multiple sclerosis in a subject, comprising administering to the subject a therapeutically effective amount of T cells engineered to express a chimeric antigen receptor (CAR) that specifically binds C-type lectin-like molecule-1 (CLL-1, CLEC12A). Aspect 2: A method of treating multiple sclerosis in a subject, comprising administering to the subject an engineered T cell that specifically binds to CLL-1 under conditions effective to deplete monocyte-derived macrophages that infiltrate the central nervous system via CCR2+ monocyte recruitment, thereby reducing accumulation of lymphocytes in perivascular spaces. Aspect 3: A method of treating a CLL-1-expressing myeloid cell-mediated autoimmune disease or disorder in a subject, comprising administering to the subject a therapeutically effective amount of engineered CAR-T cells that express a chimeric antigen receptor (CAR) targeting C-type lectin-like molecule-1 (CLL-1), wherein the CAR-T cells target CLL-1-expressing border-associated macrophages. Aspect 4: A method of monitoring response to a CLL-1-targeted treatment therapy in a subject with multiple sclerosis, comprising measuring calprotectin levels in cerebrospinal fluid (CSF) before and after administration of CAR-T cells targeting CLL-1. Aspect 5: A method of treating an autoimmune neurological disease in a subject, comprising administering to the subject a therapeutically effective amount of engineered CAR-T cells that express a chimeric antigen receptor (CAR) targeting C-type lectin-like molecule-1 (CLL-1), wherein the CAR-T cells target CLL-1-expressing border-associated macrophages. Aspect 6: A method of reducing neuroinflammation in a subject, comprising administering to the subject a therapeutically effective amount of engineered CAR-T cells that express a chimeric antigen receptor (CAR) targeting C-type lectin-like molecule-1 (CLL-1). Aspect 7: A method of treating multiple sclerosis in a subject, comprising depleting border-associated macrophages in the subject by administering engineered CAR-T cells that express a chimeric antigen receptor targeting C-type lectin-like molecule-1 (CLL-1).

Aspect 8: The method of any one of Aspects 1 to 7, wherein the CLL-1-expressing myeloid cells are border-associated macrophages. Aspect 9: The method of any one of Aspects 1 to 8, wherein depletion of CLL-1-expressing myeloid cells reduces compartmentalized neuroinflammation or demyelination. Aspect 10: The method of any one of Aspects 1 to 9, wherein the engineered T cells traffic into the central nervous system via the choroid plexus. Aspect 11: The method of any one of Aspects 1 to 10, wherein the subject is selected for treatment based on elevated cerebrospinal fluid calprotectin. Aspect 12: The method of any one of Aspects 1 to 11, wherein the subject has progressive multiple sclerosis. Aspect 13: The method of any one of Aspects 1 to 12, wherein the engineered T cells are autologous CAR-T cells. Aspect 14: The method of any one of Aspects 1 to 13, wherein the engineered T cells are allogeneic CAR-T cells and comprise a disruption of TRAC and/or B2M. Aspect 15: The method of any one of Aspects 1 to 14, wherein the engineered T cells are allogeneic CAR-T cells and further comprise a T-cell receptor knockout, a programmed cell death protein-1 knockout, and a B2M knockout with an HLA-E knock-in. Aspect 16: The method of any one of Aspects 1 to 15, wherein the engineered T cells are allogeneic CAR-T cells and are formulated for intravenous infusion.

Aspect 17: The method of any one of Aspects 2 to 16, wherein the reduction of perivascular lymphocytes occurs independently of blood-brain barrier disruption. Aspect 18: The method of any one of Aspects 2 to 17, wherein the perivascular lymphocytes accumulate through trafficking of interstitial fluid derived from fenestrated epithelia. Aspect 19: The method of Aspect 18, wherein the fenestrated epithelia comprise the choroid plexus. Aspect 20: The method of Aspect 18, wherein the fenestrated epithelia comprise the dura mater. Aspect 21: The method of Aspect 18, wherein the fenestrated epithelia comprise the leptomeninges. Aspect 22: The method of Aspect 18, wherein depletion of monocyte-derived macrophages alters the local interstitial fluid milieu to prevent perivascular lymphocyte accumulation. Aspect 23: The method of any one of Aspects 2 to 22, wherein the perivascular lymphocytes are selected from the group consisting of T cells and B cells. Aspect 24: The method of any one of Aspects 2 to 23, wherein the reduction of perivascular lymphocytes correlates with improvement in progressive multiple sclerosis.

Aspect 25: The method of any one of Aspects 1 to 24, wherein the CAR-T cells comprise at least one modification selected from the group consisting of T cell receptor knockout, programmed cell death protein 1 knockout, beta-2-microglobulin knockout, and B2M-HLA-E insertion. Aspect 26: The method of Aspect 25, wherein the CAR-T cells comprise a T cell receptor knockout, a programmed cell death protein 1 knockout, a beta-2-microglobulin knockout, and a B2M-HLA-E insertion. Aspect 27: The method of any one of Aspects 1 to 26, further comprising administering to the subject a lymphodepletion regimen prior to administration of the CAR-T cells. Aspect 28: The method of Aspect 27 or Aspect 4, wherein the lymphodepletion regimen comprises fludarabine and cyclophosphamide. Aspect 29: The method of Aspect 3 or Aspect 5, wherein the CLL-1-expressing myeloid cell-mediated autoimmune disease or disorder is multiple sclerosis. Aspect 30: The method of Aspect 6 or Aspect 12, wherein the multiple sclerosis is selected from the group consisting of relapsing-remitting multiple sclerosis, secondary progressive multiple sclerosis, and primary progressive multiple sclerosis.

Aspect 31: The method of any one of Aspects 1 to 30, further comprising monitoring the subject for a response to the CAR-T cells by measuring calprotectin levels in cerebrospinal fluid. Aspect 32: The method of Aspect 9, wherein the border-associated macrophages are choroid plexus macrophages. Aspect 33: The method of Aspect 9 or Aspect 25, wherein the CAR-T cells comprise at least one modification selected from the group consisting of T cell receptor knockout, programmed cell death protein 1 knockout, beta-2-microglobulin knockout, and B2M-HLA-E insertion.

Aspect 34: The method of Aspect 9, Aspect 29 or Aspect 37, wherein the CLL-1-expressing myeloid cell-mediated autoimmune disease or disorder is multiple sclerosis. Aspect 35: The method of Aspect 12 or Aspect 30, wherein the multiple sclerosis is selected from the group consisting of relapsing-remitting multiple sclerosis, secondary progressive multiple sclerosis, and primary progressive multiple sclerosis. Aspect 36: The method of Aspect 14, wherein smoldering neuroinflammation is mediated by CLL-1-expressing border-associated macrophages. Aspect 37: The method of Aspect 14, Aspect 29 or Aspect 34, wherein the CLL-1-expressing myeloid cell-mediated autoimmune disease or disorder is multiple sclerosis. Aspect 38: The method of Aspect 16 or Aspect 30, wherein the multiple sclerosis is selected from the group consisting of relapsing-remitting multiple sclerosis, secondary progressive multiple sclerosis, and primary progressive multiple sclerosis. Aspect 39: The method of any one of Aspects 1 to 38, wherein the subject has or is suspected of having an autoimmune disorder, an inflammatory disorder, a neurologic or neurodegenerative disease, or an Epstein-Barr virus (EBV)-associated lymphoproliferative disease. Aspect 40: The method of any one of Aspects 1 to 39, wherein the subject has or is suspected of having an autoimmune disease. Aspect 41: The method of Aspect 40, wherein the autoimmune disease is selected from the group consisting of Achalasia, Addison's disease, Adult Still's disease, Agammaglobulinemia, Alopecia arcata, Amyloidosis, Ankylosing spondylitis, Anti-GBM nephritis, Anti-TBM nephritis, Anti-MOG antibody-associated disease, Antiphospholipid syndrome, Autism, Autoimmune angioedema, Autoimmune dysautonomia, Autoimmune encephalomyelitis, Autoimmune hepatitis, Autoimmune inner car disease, Autoimmune myocarditis, Autoimmune oophoritis, Autoimmune orchitis, Autoimmune pancreatitis, Autoimmune retinopathy, Autoimmune urticaria, Axonal and neuronal neuropathy, Bal6 disease, Behcet's disease, Benign mucosal pemphigoid, Bullous pemphigoid, Castleman disease, Celiac disease, Chagas disease, Chronic inflammatory demyelinating polyneuropathy, Chronic recurrent multifocal osteomyelitis, Churg-Strauss syndrome, Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease, Discoid lupus, Dressler's syndrome, Endometriosis, Eosinophilic esophagitis, Eosinophilic fasciitis, Erythema nodosum, Essential mixed cryoglobulinemia, Evans syndrome, Fibromyalgia, Fibrosing alvcolitis, Giant cell arteritis, Giant cell myocarditis, Glomerulonephritis, Goodpasture's syndrome, Granulomatosis with polyangiitis, Graves' disease, Guillain-Barre syndrome, Hashimoto's thyroiditis, Hemolytic anemia, Henoch-Schonlein purpura, Herpes gestationis, Hidradenitis suppurativa, Hypogammaglobulinemia, IgA nephropathy, IgG4-related sclerosing disease, Immune thrombocytopenia purpura, Inclusion body myositis, Interstitial cystitis, Juvenile arthritis, Juvenile diabetes, Juvenile myositis, Kawasaki disease, Lambert-Eaton syndrome, Leukocytoclastic vasculitis, Lichen planus, Lichen sclerosus, Ligneous conjunctivitis, Lincar IgA disease, Lupus, Lyme disease chronic, Meniere's disease, Microscopic polyangiitis, Mixed connective tissue disease, Mooren's ulcer, Mucha-Habermann disease, Multifocal motor neuropathy, Multiple sclerosis, Myasthenia gravis, Myositis, Narcolepsy, Neonatal lupus, Neuromyelitis optica, Neutropenia, Ocular cicatricial pemphigoid, Optic neuritis, Palindromic rheumatism, PANDAS, Paraneoplastic cerebellar degeneration, Paroxysmal nocturnal hemoglobinuria, Parry Romberg syndrome, Pars planitis, Parsonage-Turner syndrome, Pemphigus, Peripheral neuropathy, Perivenous encephalomyelitis, Pernicious anemia, POEMS syndrome, Polyarteritis nodosa, Polyglandular syndromes, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia, Pyoderma gangrenosum, Raynaud's phenomenon, Reactive arthritis, Reflex sympathetic dystrophy, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm and testicular autoimmunity, Stiff person syndrome, Subacute bacterial endocarditis, Susac's syndrome, Sympathetic ophthalmia, Takayasu's arteritis, Temporal arteritis, Thrombocytopenia purpura, Tolosa-Hunt syndrome, Transverse myelitis, Type I diabetes, Ulcerative colitis, Undifferentiated connective tissue disease, Uveitis, Vasculitis, Vitiligo, and Vogt-Koyanagi-Harada Disease. Aspect 42: The method of any one of Aspects 1 to 41, wherein the subject has or is suspected of having an inflammatory disease. Aspect 43: The method of Aspect 42, wherein the inflammatory disease is selected from the group consisting of allergy, ankylosing spondylitis, asthma, atopic dermatitis, autoimmune diseases or disorders, celiac disease, chronic obstructive pulmonary disease, chronic peptic ulcer, cystic fibrosis, diabetes, Familial Mediterranean fever, glomerulonephritis, gout, hepatitis, inflammatory bowel disease, myositis, osteoarthritis, pelvic inflammatory disease, multiple sclerosis, neurodegenerative diseases of aging, periodontal disease, pre-perfusion injury transplant rejection, progressive rubella panencephalitis, psoriasis, rheumatic disease, scleroderma, sinusitis, subacute sclerosing panencephalitis, and tuberculosis. Aspect 44: The method of any one of Aspects 1 to 43, wherein the subject has or is suspected of having a neurological or neurodegenerative disease. Aspect 45: The method of Aspect 44, wherein the neurological or neurodegenerative disease is selected from the group consisting of attention-deficit disorder, attention-deficit hyperactivity disorder, Asperger syndrome, autism spectrum disorder, amyotrophic lateral sclerosis, Alzheimer's disease, Brown-Sequard syndrome, cerebral palsy, Guillain-Barre syndrome, Gaucher disease, Huntington's disease, multiple sclerosis, muscular dystrophy, and spinal muscular atrophy. Aspect 46: The method of any one of Aspects 1 to 45, wherein the subject has or is suspected of having a white matter inflammatory or demyelinating disease. Aspect 47: The method of Aspect 46, wherein the white matter inflammatory or demyelinating disease is selected from the group consisting of multiple sclerosis, neuromyelitis optica spectrum disorder, myelin oligodendrocyte glycoprotein antibody-associated disease, acute disseminated encephalomyelitis, optic neuritis, transverse myelitis, Balo's concentric sclerosis, Schilder's disease, Marburg's variant multiple sclerosis, tumefactive demyelinating disease, Susac's syndrome, anti-aquaporin-4 antibody-associated disorders, anti-MOG antibody-associated disorders, progressive multifocal leukoencephalopathy, leukodystrophies with inflammatory features, post-infectious or post-vaccination demyelination, and disorders characterized by immune-mediated injury to central nervous system white matter. Aspect 48: The method of any one of Aspects 1 to 47, wherein the subject has or is suspected of having an Epstein-Barr virus-associated lymphoproliferative disease. Aspect 49: The method of Aspect 48, wherein the Epstein-Barr virus-associated lymphoproliferative disease is selected from the group consisting of EBV-associated reactive lymphoid proliferations, EBV-positive reactive lymphoid hyperplasia, infectious mononucleosis, EBV-related hemophagocytic lymphohistiocytosis, chronic active EBV infection, severe mosquito bite allergy, hydroa vacciniforme-like lymphoproliferative disease, EBV-positive mucocutaneous ulcer, non-Hodgkin's lymphomas, EBV-positive B cell lymphoproliferative diseases, EBV-positive Burkitt lymphoma, lymphomatoid granulomatosis, Hodgkin lymphoma, diffuse large B cell lymphoma, EBV-associated diffuse large B cell lymphoma associated with chronic inflammation, fibrin-associated diffuse large B cell lymphoma, human herpes virus 8-associated B cell lymphoproliferative disorders, primary effusion lymphoma, germinotropic lymphoproliferative disorder, EBV-positive plasmablastic lymphoma, plasma cell myeloma, EBV-positive NK/T cell lymphoproliferative diseases, peripheral T-cell lymphomas, extranodal NK/T cell lymphoma, nasal type, peripheral T cell lymphoma, angioimmunoblastic T cell lymphoma, follicular T cell lymphoma, systemic EBV-positive T cell lymphoma of childhood, EBV-associated aggressive NK cell leukemia, intravascular NK/T-cell lymphomas, immunodeficiency-related lymphoproliferative disorders, HIV-related lymphoproliferative disease, post-transplant lymphoproliferative disorders, EBV-associated histiocytic-dendritic disorders, and inflammatory pseudotumor-like follicular/fibroblastic dendritic cell sarcoma. Aspect 50: The method of any one of Aspects 1 to 49, wherein the target antigen for the engineered T cell is a C-type lectin family member expressed on monocyte-derived or disease-associated macrophages that infiltrate the central nervous system and replace yolk sac-derived border-associated macrophages. Aspect 51: The method of Aspect 50, wherein the C-type lectin family member is selected from CLEC1 through CLEC16, CLEC2, CLEC2A, CLEC2B, CLEC4A through CLEC4G, CLEC5A, CLEC6A, CLEC7A, CLEC9A, CLEC10A, CLECIIA, CLEC12A, CLEC14A, CLEC16A, and functionally similar lectins selectively upregulated on pathogenic macrophages.

Aspect 52: A kit for treating multiple sclerosis in a subject, comprising: a) an engineered T cell configured to specifically bind to C-type lectin-like molecule-1 (CLL-1); and (b) instructions for administration comprising co-administration of a non-myelosuppressive lymphodepleting agent, the lymphodepleting agent comprising an anti-CD20 monoclonal antibody, prior to administration of the engineered T cell, under conditions effective to reduce accumulation of pathogenic B cells in perivascular spaces, wherein the co-administration avoids recruitment of CCR2-positive monocytic myeloid-derived suppressor cells that in oncology provide pro-survival signals to malignant B cells.

Aspect 53: The kit of Aspect 52, wherein the engineered T cell comprises a chimeric antigen receptor (CAR) targeting CLL-1. Aspect 54: The kit of Aspect 53, wherein the engineered T cell is a CAR-T cell selected from autologous CAR-T cells or allogeneic CAR-T cells. Aspect 55: The kit of Aspect 54, wherein the CAR-T cell comprises at least one genome edit selected from T cell receptor knockout, programmed cell death protein 1 knockout, beta-2-microglobulin knockout, or B2M-HLA-E insertion. Aspect 56: The kit of any one of Aspects 52 to 55, wherein the CAR-T cell comprises a transmembrane and intracellular signaling domain comprising CD35, and a costimulatory domain selected from the group consisting of 4-1BB, CD28, and OX40. Aspect 57: The kit of any one of Aspects 52 to 56, wherein the lymphodepleting agent comprises an anti-CD20 monoclonal antibody selected from the group consisting of rituximab, ocrelizumab, ofatumumab, or obinutuzumab. Aspect 58: The kit of any one of Aspects 52 to 57, further comprising a pharmaceutical carrier. Aspect 59: The kit of any one of Aspects 52 to 58, wherein the instructions further comprise administration of an agent selected from fludarabine, cyclophosphamide, or a combination thereof. Aspect 60: The kit of any one of Aspects 52 to 59, further comprising one or more vials of engineered T cells, one or more vials of anti-CD20 monoclonal antibody, and a container or packaging identifying the kit for treatment of multiple sclerosis. Aspect 61: The kit of any one of Aspects 52 to 60, wherein the instructions specify that the lymphodepleting agent is administered from 1 to 7 days before administration of the engineered T cell. Aspect 62: The kit of any one of Aspects 52 to 61, wherein the kit is configured for use in a subject selected based on elevated cerebrospinal fluid calprotectin or for a subject with progressive multiple sclerosis. Aspect 63: The kit of any one of Aspects 52 to 62, wherein the engineered T cell further comprises at least one modification selected from reduction of checkpoint inhibitors selected from PD-1, CTLA-4, LAG-3, or TIM-3. Aspect 64: The kit of any one of Aspects 52 to 63, wherein the instructions are printed, electronic, or encoded as a digital file or QR code.

Aspect 65: A system for treating multiple sclerosis in a subject, comprising: (a) an engineered T cell configured to specifically bind to CLL-1; (b) a delivery apparatus for CAR-T cell infusion; (c) a sampling apparatus for obtaining cerebrospinal fluid or biological fluid; (d) an analysis unit configured to measure calprotectin levels; and (c) a controller configured to implement dosing and monitoring protocols, wherein the system is configured so that administration of a non-myelosuppressive lymphodepleting agent comprising an anti-CD20 monoclonal antibody occurs prior to administration of the engineered T cell, and wherein the system is further configured such that said co-administration is effective to reduce accumulation of pathogenic B cells in perivascular spaces and to avoid recruitment of CCR2-positive monocytic myeloid-derived suppressor cells.

Aspect 66: The system of Aspect 65, further comprising a display, database of reference biomarker levels, or module configured to integrate biomarker data with clinical decision support. Aspect 67: The system of any one of Aspects 65 to 66, wherein the analysis unit is configured to measure calprotectin by immunoassay. Aspect 68: The system of any one of Aspects 65 to 67, wherein the controller is configured to prompt a go/no-go decision for further therapy based on measured calprotectin. Aspect 69: The system of any one of Aspects 65 to 68, wherein the delivery apparatus is configured to infuse the engineered T cell formulated for intravenous delivery, and the subject is selected for treatment based on disease severity or calprotectin level. Aspect 70: The kit or system of any one of Aspects 52 to 69, wherein the engineered T cell is a CAR-T cell that is autologous or allogeneic. Aspect 71: The kit or system of any one of Aspects 52 to 70, wherein the subject has progressive multiple sclerosis.

1. A method of treating multiple sclerosis in a subject, comprising administering to the subject a therapeutically effective amount of T cells engineered to express a chimeric antigen receptor (CAR) that specifically binds C-type lectin-like molecule-1 (CLL-1, CLEC12A), thereby treating the multiple sclerosis.

2. The method of claim 1, wherein the CLL-1-expressing myeloid cells are monocyte derived macrophages.

3. The method of claim 1, wherein depletion of CLL-1-expressing myeloid cells reduces compartmentalized neuroinflammation or demyelination.

4. The method of claim 1, wherein the engineered T cells traffic into the central nervous system via the choroid plexus.

5. The method of claim 1, wherein the subject is selected for treatment based on elevated cerebrospinal fluid calprotectin.

6. The method of claim 1, wherein the subject has progressive multiple sclerosis.

7. The method of claim 1, wherein the engineered T are autologous CAR-T cells.

8. The method of claim 1, wherein the engineered T are allogeneic CAR-T cells and comprise a disruption of TRAC or B2M, or a combination thereof.

9. The method of claim 1, wherein the engineered T cells are allogeneic CAR-T cells and further comprise a T-cell receptor (TCR) knockout, a PD-1 knockout, and a B2M knockout with an HLA-E knock-in.

10. The method of claim 1, wherein the engineered T cells are allogeneic CAR-T cells and are formulated for intravenous infusion.

11. A method of treating multiple sclerosis in a subject, comprising administering to the subject an engineered T cell that specifically binds to CLL-1 under conditions effective to deplete monocyte-derived macrophages that infiltrate the central nervous system via CCR2+ monocyte recruitment, thereby reducing accumulation of lymphocytes in perivascular spaces.

12. The method of claim 11, wherein the reduction of perivascular lymphocytes occurs independently of blood-brain barrier disruption.

13. The method of claim 11, wherein the perivascular lymphocytes accumulate through trafficking of interstitial fluid derived from fenestrated epithelia.

14. The method of claim 13, wherein the fenestrated epithelia comprise the choroid plexus.

15. The method of claim 13, wherein depletion of monocyte-derived macrophages alters the local interstitial fluid milieu to prevent perivascular lymphocyte accumulation.

16. The method of claim 11, wherein the perivascular lymphocytes are selected from the group consisting of T cells and B cells.

17. The method of claim 11, wherein the reduction of perivascular lymphocytes correlates with improvement in progressive multiple sclerosis.

18. A system for treating multiple sclerosis in a subject, comprising:
an engineered T cell that specifically binds to CLL-1; and
instructions for co-administration of a non-myelosuppressive lymphodepleting agent, the lymphodepleting agent comprising an anti-CD20 monoclonal antibody, prior to administration of the engineered T cell, under conditions effective to reduce accumulation of pathogenic B cells in perivascular spaces, wherein said co-administration avoids recruitment of CCR2+ monocytic myeloid-derived suppressor cells that in oncology provide pro-survival signals to malignant B cells.

19. The system of claim 18, wherein the anti-CD20 monoclonal antibody is selected from the group consisting of ofatumumab, ocrelizumab, rituximab, and ublituximab.

20. The system of claim 18, wherein the lymphodepleting agent further comprises cladribine.

21. The system of claim 18, wherein the lymphodepleting agent further comprises alemtuzumab.

22. The system of claim 18, wherein the lymphodepleting agent further comprises mitoxantrone.

23. The system of claim 18, wherein reduction of pathogenic B cells in perivascular spaces correlates with decreased meningeal inflammation.

24. The system of claim 18, wherein the instructions specify use in subjects diagnosed with progressive multiple sclerosis.
